(19) United States
(12) Patent Application Publication
(10) Pub. No.: US 2023/0211001 A1
(43) Pub. Date: Jul. 6, 2023

(54) Lysosomal Targeting Molecules Comprising Knottin Peptides And Related Compositions And Methods

(71) Applicant: The Board of Trustees of the Leland Stanford Junior University, Stanford, CA (US)

(72) Inventors: Green Ahn, Stanford, CA (US); Caitlyn Miller, Standford, CA (US); Carolyn R. Bertozzi, Menlo Park, CA (US); Jennifer R. Cochran, Stanford, CA (US); Steven Banik, Redwood City, CA (US)

(21) Appl. No.: 18/018,238

(22) PCT Filed: Jul. 30, 2021
(86) PCT No.: PCT/US2021/044027

§ 371 Date: Jan. 26, 2023

Related U.S. Application Data
(60) Provisional application No. 63/059,010, filed on Jul. 30, 2020.

(51) Int. Cl. A61K 47/64; A61P 35/00

(52) U.S. Cl. CPC A61K 47/6425; A61K 47/6415; A61P 35/00

(57) ABSTRACT

Provided are bifunctional molecules that include a first moiety that specifically binds a cell surface molecule, and a second moiety that specifically binds a lysosomal targeting molecule. In certain embodiments, the first moiety is a knottin peptide comprising an engineered loop that binds to the cell surface molecule. The bifunctional molecules find use, e.g., for targeted degradation of cell surface molecules (e.g., proteins) via the endosomal/lysosomal pathway. Also provided are compositions and kits that include the bifunctional molecules, as well as methods of using the bifunctional molecules. Methods of making bifunctional molecules are also provided.

### CROSS-REFERENCE TO RELATED APPLICATIONS

This application claims the benefit of U.S. Provisional Patent Application No. 63/059,010, filed Jul. 30, 2020, which application is incorporated herein by reference in its entirety.

### STATEMENT OF GOVERNMENT SUPPORT

This invention was made with Government support under contracts CA049605 and GM058867 awarded by the National Institutes of Health. The Government has certain rights in the invention.

### INTRODUCTION

Targeted protein degradation (TPD) is a promising new therapeutic modality and a tool for probing biological pathways as well as the cellular degradation machinery. Most TPD platforms such as IMiDs, proteolysis targeting chimeras (PROTACs), dTAGs, Trim-Away, and SNIPERs, rely on the ubiquitin proteasome system (UPS). Recently, AUTACs and ATTECs have emerged as approaches that exploit the intracellular autophagy machinery for mediating TPD. Advances in TPD strategies have accelerated over the past two decades, and PROTACs have recently entered clinical trials. However, the cytosolic localization of the UPS and targetable autophagy machinery restricts these approaches to proteins with cytosolic domains and requires degraders to be cell-permeable.

Cystine-knot miniproteins, also known as knottins, are a structural family (typically 30-50 amino acids in length) characterized by a core of antiparallel p-strands stabilized by at least three disulfide bonds. In a characteristic cystine-knot motif, the first and fourth and the second and fifth cysteine residues form disulfide bonds. A disulfide bond formed between the third and sixth cysteine residues passes through these first two disulfides, creating a macrocyclic knot. This disulfide-constrained core confers chemical, thermal and proteolytic stability upon the peptide.

Knottins also possess loop regions of variable length and composition that are constrained to the core of antiparallel p-strands. These loops are important for folding, structural integrity, molecular recognition and biological function. The loop regions of knottin peptides have been shown to tolerate amino acid mutations. In contrast to linear peptides, knottins have been shown to retain their three-dimensional structure and function after boiling or incubation in acid, base and serum.

Polypeptides containing cysteine-knot motifs are found in a variety of fungi, plants and animals, and carry out diverse functions including protease inhibition, ion channel blockade and antimicrobial activity. As examples, toxins from scorpions, spiders and snails block ion channel activity, the Ecballium elaterium trypsin inhibitor II (EETI-II) from plants inhibits serine proteases and the human Agouti-Related Protein (AgRP) is a regulatory neuropeptide.

### SUMMARY

Provided are bifunctional molecules that include a first moiety that specifically binds a cell surface molecule, and a second moiety that specifically binds a lysosomal targeting molecule. In certain embodiments, the first moiety is a knottin peptide comprising an engineered loop that binds to the cell surface molecule. The bifunctional molecules find use, e.g., for targeted degradation of cell surface molecules (e.g., proteins) via the endosomal/lysosomal pathway. Also provided are compositions and kits that include the bifunctional molecules, as well as methods of using the bifunctional molecules. Methods of making bifunctional molecules are also provided.

### BRIEF DESCRIPTION OF THE FIGURES

FIG. 1 A schematic illustration of a bifunctional molecule and uses thereof according to embodiments of the present disclosure.

FIG. 2 A scheme for synthesizing mannose-6-phosphate N-carboxyanhydride according to one embodiment. This route allows access to mannose-6-phosphate glycans linked to, e.g., serine residues for use as monomers in N-carboxyanhydride polymerization.

FIG. 3 A scheme for synthesizing mannose-6-phosphonate N-carboxyanhydride according to one embodiment. This route allows access to mannose-6-phosphonate glycans linked to, e.g., serine residues for use as monomers in N-carboxyanhydride polymerization. The phosphonate group is a hydrolytically stable variant of a phosphate group which has previously demonstrated better serum stability compared to mannose-6-phosphate glycans.

FIG. 4 A scheme for synthesizing mannose-6-carboxylate N-carboxyanhydride according to one embodiment. This route allows access to mannose-6-carboxylate glycans linked to, e.g., serine residues for use as monomers in N-carboxyanhydride polymerization. The carboxylate group is a hydrolytically stable variant of a phosphate group which has previously demonstrated better serum stability compared to mannose-6-phosphate glycans. Mannose-6-carboxylate glycans have previously demonstrated a relative binding affinity of 0.3 for the cation independent M6PR (CIM6PR), compared to mannose-6-phosphate glycans. The ability to chemically tune the receptor-ligand interaction allows for greater control in biological applications to diminish off-target binding events.

FIG. 5 A scheme for synthesizing mannose-6-acrylate N-carboxyanhydride according to one embodiment. This route allows access to mannose-6-acrylate glycans linked to, e.g., serine residues for use as monomers in N-carboxyanhydride polymerization. The acrylate group is a hydrolytically stable variant of a phosphate group which has previously demonstrated better serum stability compared to mannose-6-phosphate glycans. Mannose-6-acrylate glycans have previously demonstrated a relative binding affinity of 0.7 for the CIM6PR, compared to mannose-6-phosphate glycans. The ability to chemically tune the receptor-ligand interaction allows for greater control in biological applications to diminish off-target binding events.

FIG. 6 A scheme for synthesizing glucose-6-phosphonate N-carboxyanhydride according to one embodiment. This route allows access to glucose-6-phosphonate glycans linked to, e.g., serine residues for use as monomers in N-carboxyanhydride polymerization. The glucose-6-phosphonate residue has a significantly weaker binding affinity for the CIM6PR compared to mannose-containing glycans.

FIG. 7 A scheme for synthesizing mannose-6-phosphonate isothiocyanate according to one embodiment. This route allows access to mannose-6-phosphonate isothiocyanate (M6Pn-ITC), which can be directly conjugated to, e.g., lysine residues in proteins. Conjugation of multiple M6Pn-ITC to multiple amino acids (e.g., lysines) within a given protein allows for multivalent presentation of M6Pn glycans.

FIG. 8 An illustration of a general NCA polymerization scheme for synthesizing a scaffold for displaying M6P ligands according to one embodiment. Copolymers with other amino-acid derived NCAs are readily synthesized similarly, and provide access to numerous polymers with varied structures and compositions bearing multiple M6P ligand residues. These materials are subsequently deprotected to provide the full polypeptide/glycan structure.

FIG. 9 A scheme for solid-phase peptide synthesis of mannose-6-phosphonate peptide oligomer according to one embodiment. As shown, a scaffold for displaying M6P ligands can also be synthesized using solid-phase peptide synthesis, starting from M6P, M6Pn, etc. amino acids. This synthetic route allows for greater control over polypeptide length and composition compared to the NCA polymerization route, and does not require special synthesis conditions compared to the NCA polymerization-derived materials.

FIG. 10 A schematic illustration (top) and fluorescence imaging results (bottom) of an experiment demonstrating that M6Pn polymers which have been functionalized with a biotin cap can mediate transfer of extracellular NeutrAvidin-647 (NA647—a protein to which biotin strongly binds) to lysosomes from the extracellular space for degradation. Colocalization of both protein and lysosome staining dye are observed.

FIG. 11 Data demonstrating that several cell lines exhibit uptake of NA647 in a M6Pn polymer dependent manner. In view of these results, it is expected that any cell line bearing M6PRs (e.g., CIM6PRs) will allow for shuttling of proteins to lysosome by this method, and is not limited to the cell lines tested in the present study.

FIG. 12 Schematic illustration of a lysosomal targeting molecule to which the second moiety of a bifunctional molecule may bind according to embodiments of the present disclosure. In this example, the lysosomal targeting molecule is asialoglycoprotein receptor (ASGPR) which is expressed substantially exclusively on liver cells, e.g., hepatocytes. As shown on the right, ASGPR constitutively recycles between the plasma membrane and the endosome, thereby bringing extracellular glycoproteins inside the cell for degradation in the lysosome. Shown on the lower left is an example bifunctional molecule comprising a first moiety that is a knottin and a second moiety comprising an ASGPR ligand for binding to ASGPR. In this example, the second moiety comprised acetylgalactosamine (GaINAc).

FIG. 13 Panel A: Synthesis of PIP—GaINAc. Tri-GaINAc-DBCO was conjugated to PIP, a knottin peptide that binds to multiple integrins. Panel B: Degradation of cell surface integrins in HEPG2 cells as determined by live-cell flow cytometry following 44 h of treatment with 100 nM PIP or PIP—GaINAc. Anti-αvβ3, anti-αvβ5 and PIP-Fc fusion construct were used for detection. PIP was genetically fused to the Fc domain of a mouse IgG2a to generate a PIP-Fc fusion construct that measures the surface expression of integrins recognized by PIP. Panel C: Percent proliferation of HEPG2 cells following 44 h of treatment with 50 nM, 100 nM or 200 nM PIP or PIP—GaINAc. Proliferation was quantified by phase confluence over time using IncuCyte. Panel D: Time course of percent proliferation of HEPG2 cells during 44 h of treatment with 200 nM PIP or PIP—GaINAc. Panel E: Percent proliferation of HEPG2 cells for 6 d following wash-out. Cells were treated with 200 nM PIP or PIP—GaINAc on day 0. PIP+ and PIP—GaINAc+ indicate the conditions where cells were washed on day 4 and replaced with fresh medium without treatment. PIP++ and PIP—GaINAc++ indicate the conditions where cells were washed on day 4 and replaced with fresh medium containing 200 nM PIP or PIP—GaINAc. Panel F: Live HEPG2 imaging by IncuCyte throughout the 5 d after treatment with 100 nM PIP or PIP—GaINAc. Images are representative of three independent experiments for F. Data in B-E represent three independent experiments, where B and C show the mean of three independent experiments ±s.e.m. P values were determined by unpaired two-tailed t-tests.

FIG. 14 Peptide sequence of PIP and ESI-MS of PIP and PIP—GaINAc. Panel A: Peptide sequence of 33-mer PIP knottin (SEQ ID NO:42). Sequence in blue represents the binding loop with the integrin binding domain underlined, and yellow brackets represent disulfide bridges. 5-azido-L-norvaline was incorporated as a click handle. Panel B: Deconvoluted ESI-MS spectrum and total ion chromatogram of PIP. Panel C: Deconvoluted ESI-MS spectrum and total ion chromatogram of PIP—GaINAc. Calibration mix (ESI-L Low Concentration Tuning Mix Agilent P/N G1969-85000) was infused during the LC divert step.

### DETAILED DESCRIPTION

Provided are bifunctional molecules that include a first moiety that specifically binds a cell surface molecule, and a second moiety that specifically binds a lysosomal targeting molecule. In certain embodiments, the first moiety is a knottin peptide comprising an engineered loop that binds to the cell surface molecule. The bifunctional molecules find use, e.g., for targeted degradation of cell surface molecules (e.g., proteins) via the endosomal/lysosomal pathway. Also provided are compositions and kits that include the bifunctional molecules, as well as methods of using the bifunctional molecules. Methods of making bifunctional molecules are also provided.

Before the bifunctional molecules, compositions, kits and methods of the present disclosure are described in greater detail, it is to be understood that the bifunctional molecules, compositions, kits and methods are not limited to particular embodiments described, as such may, of course, vary. It is also to be understood that the terminology used herein is for the purpose of describing particular embodiments only, and is not intended to be limiting, since the scope of the bifunctional molecules, compositions, kits and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the bifunctional molecules, compositions, kits and methods. The upper and lower limits of these smaller ranges may independently be included in the smaller ranges and are also encompassed within the bifunctional molecules, compositions, kits and methods, subject to any specifically excluded limit in the stated range. Where the stated range includes one or both of the limits, ranges excluding either or both of those included limits are also included in the bifunctional molecules, compositions, kits and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the bifunctional molecules, compositions, kits and methods belong. Although any bifunctional molecules, compositions, kits and methods similar or equivalent to those described herein can also be used in the practice or testing of the bifunctional molecules, compositions, kits and methods, representative illustrative bifunctional molecules, compositions, kits and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present bifunctional molecules, compositions, kits and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the bifunctional molecules, compositions, kits and methods, which are, for clarity, described in the context of separate embodiments, may also be provided in combination in a single embodiment. Conversely, various features of the bifunctional molecules, compositions, kits and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present bifunctional molecules, compositions, kits and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Bifunctional Molecules

The present disclosure provides bifunctional molecules that include a first moiety that specifically binds a cell surface molecule or extracellular molecule, and a second moiety that specifically binds a lysosomal targeting molecule. In certain embodiments, the first moiety is a knottin peptide comprising an engineered loop that binds to the cell surface molecule or extracellular molecule. According to some embodiments, the first moiety specifically binds one or more integrins. Certain non-limiting embodiments of the bifunctional molecules will now be described.

Knottin Peptides

When the first moiety is a knottin peptide, the type of knottin peptide employed in the bifunctional molecule may vary. The three-dimensional structure of a knottin peptide is minimally defined by a particular arrangement of three disulfide bonds. This characteristic topology forms a molecular knot in which one disulfide bond passes through a macrocycle formed by the other two intra-chain disulfide bridges. Although their secondary structure content is generally low, knottins share a small triple-stranded antiparallel p-sheet, which is stabilized by the disulfide bond framework. Folding and functional activity of knottins are often mediated by loop regions that are diverse in both length and amino acid composition. While three disulfide bonds are the minimum number that defines the fold of this family of peptides, knottins can also contain additional cysteine residues, yielding molecules with four or more disulfide bonds and additional constrained loops in their structure. The term “cystine” refers to a Cys residue in which the sulfur group is linked to another amino acid though a disulfide linkage; the term “cysteine” refers to the —SH (“half cystine”) form of the residue. Binding loop portions may be adjacent to cystines, such that there are no other intervening cystines in the primary sequence in the binding loop.

The knottin peptide may be a peptide described in the online KNOTTIN database, which includes detailed amino acid sequence, structure, classification and function information for thousands of polypeptides identified as contain cystine-knot motifs. Knottins are found in a variety of plants, animals, insects and fungi.

The knottin peptide may be full-length (that is, the length of the wild-type peptide/polypeptide), the knottin peptide may be truncated relative to the length of the wild-type peptide/polypeptide, or the knottin peptide may include additional amino acids such that the peptide is greater in length relative to the length of the wild-type peptide/polypeptide.

According to certain embodiments, a bifunctional molecule of the present disclosure includes a knottin peptide based on any one of an Ecballium elaterium trypsin inhibitor II (EETI-II) peptide, an agouti-related protein (AgRP) peptide, a w-conotoxin peptide, a Kalata B1 peptide, an MCoTI-II peptide, an agatoxin peptide, or a chlorotoxin peptide. In some embodiments, the knottin peptide is based on an Ecballium elaterium trypsin inhibitor II (EETI-II) peptide. In some embodiments, the knottin peptide is based on an agouti-related protein (AgRP) peptide.

By “EETI” is meant Protein Data Bank Entry (PDB) 2ETI. Its entry in the KNOTTIN database is EETI-II. In certain aspects, a knottin peptide of a conjugate of the present disclosure is based on an EETI-II peptide having the following amino acid sequence:

(SEQ ID NO: 1)
GCPRILMRCKQDSDCLAGCVCGPNGFCG

By “AGRP” is meant PDB entry 1 HYK and KNOTTIN database entry SwissProt AGRP_HUMAN. AGRP is a 132 amino acid neuropeptide that binds to melanocortin receptors in the human brain and is involved in regulating metabolism and appetite. The biological activity of AgRP is mediated by its C-terminal cysteine knot domain, which contains five disulfide bonds, but a fully active 34 amino acid truncated AgRP that contains only four disulfide bonds has been developed. In certain embodiments, a knottin peptide of a bifunctional molecule of the present disclosure is based on a truncated AGRP peptide having the following amino acid sequence:

(SEQ ID NO: 2)
CVRLHESCLGQQVPCCDPAATCYCRFFNAFCYCR

According to certain embodiments, a knottin peptide of a bifunctional molecule of the present disclosure is based on a Kalata B1 peptide having the following amino acid sequence:

(SEQ ID NO: 3)
CGETCVGGTCNTPGCTCSWPVCTRNGLPV

In certain aspects, a knottin peptide of a bifunctional molecule of the present disclosure is based on a MCoTI-II peptide having the following amino acid sequence:

(SEQ ID NO: 4)
SGSDGGVCPKILKKCRRDSDCPGACICRGNGYCG

According to certain embodiments, a knottin peptide of a bifunctional molecule of the present disclosure is based on a chlorotoxin peptide having the following amino acid sequence:

(SEQ ID NO: 5)
MCMPCFTTDHQMARKCDDCCGGKGRGKCYGPQCLCR

Sequences and structural (e.g., loop) information for EETI-II, AgRP, w-conotoxin, Kalata B1, MCoTI-II, agatoxin, chlorotoxin, and other knottin peptides upon which the knottin peptides of the bifunctional molecules of the present disclosure may be based may be found in the PDB, the KNOTTIN database, and other protein databases.

The knottin peptide includes an engineered loop that binds to a cell surface molecule or extracellular molecule. Knottins contain three disulfide bonds interwoven into a molecular ‘knot’ that constrain loop regions to a core of anti-parallel β-sheets. Wild-type EETI, for example, is composed of 28 amino acids with three disulfide-constrained loops: loop 1 (the trypsin binding loop, residues 3-8), loop 2 (residues 10-14), and loop 3 (residues 22-26) Knottin family members, which include protease inhibitors, toxins, and antimicrobials, share little sequence homology apart from their core cysteine residues. As a result, their disulfide-constrained loops tolerate much sequence diversity, making knottins amenable for protein engineering applications where mutations need to be introduced into a protein without abolishing its three-dimensional fold.

The engineered loop may include amino acid substitutions, insertions, and/or deletions in an existing loop of the knottin peptide, or the engineered loop may be a loop added to the knottin protein. That is, the knottin peptide of the bifunctional molecule may include a loop in addition to the one or more loops present in the wild-type peptide. By combining directed evolution with computational covariance analysis, guidelines for introducing modifications (both in amino acid sequence and loop length) into the loop regions of the knottin scaffold have been elucidated. See, e.g., Lahti et al. (2009) PLoS Comput. Biol. 5(9): e1000499.

In certain embodiments, the knottin peptide includes one or more unnatural amino acids. Unnatural amino acids which find use for preparing the bifunctional molecules of the present disclosure include those having a functional group selected from an azide, alkyne, alkene, amino-oxy, hydrazine, aldehyde, nitrone, nitrile oxide, cyclopropene, norbornene, iso-cyanide, aryl halide, and boronic acid functional group. Unnatural amino acids which may be incorporated into a knottin peptide of a bifunctional molecule of the present disclosure, which unnatural amino acid may be selected to provide a functional group of interest are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere.

As one example, the EETI-2.5F peptide (sometimes referred to herein as “PIP”) described below may be modified to include an unnatural amino acid, e.g., having a functional group useful for attachment to a linker, e.g., a linker attached to a nucleoside drug. According to one embodiment, the unnatural amino acid replaces the serine at position 15 of EETI-2.5F (bold in the above EETI-2.5F sequence) as provided in the following amino acid sequence, where X (bold and underlined) represents an unnatural amino acid:

(SEQ ID NO: 6)
GCPRPRGDNPPLTCXQDSDCLAGCVCGPNGFCG

In certain aspects, a knottin peptide of a conjugate of the present disclosure, including but not limited to a modified EETI-2.5F peptide described herein, includes an unnatural amino acid that includes an azide functional group. An example of such an unnatural amino acid is 5-azido-L-norvaline. As demonstrated in the Experimental section below, an EETI-2.5F peptide that includes 5-azido-L-norvaline (e.g., at position 15) finds use for direct or indirect attachment to the second moiety.

The manner in which the knottin peptide has an engineered loop that binds to a cell surface or extracellular molecule may vary. Rational and combinatorial approaches have been used to engineer knottins with novel molecular recognition properties. For example, a library of knottin proteins may be created and screened, e.g., by bacterial display, phage display, yeast surface display, fluorescence-activated cell sorting (FACS), and/or any other suitable screening method.

Yeast surface display is a powerful combinatorial technology that has been used to engineer proteins with novel molecular recognition properties, increased target binding affinity, proper folding, and improved stability. In this platform, libraries of protein variants are generated and screened in a high-throughput manner to isolate mutants with desired biochemical and biophysical properties. Yeast surface display has proven to be a successful combinatorial method for engineering knottins with altered molecular recognition. Yeast surface display benefits from quality control mechanisms of the eukaryotic secretory pathway, chaperone-assisted folding, and efficient disulfide bond formation.

One example approach for developing a knottin peptide having an engineered loop that binds to a cell surface or extracellular molecule of interest involves genetically fusing the peptide to the yeast mating agglutinin protein Aga2p, which is attached by two disulfide binds to the yeast cell wall protein Aga1p. This Aga2p-fusion construct, and a chromosomally integrated Aga1p expression cassette, may be expressed under the control of a suitable promoter, such as a galactose-inducible promoter. N- or C-terminal epitope tags may be included to measure cell surface expression levels by flow cytometry using fluorescently labeled primary or secondary antibodies. This construct represents the most widely used display format, where the N-terminus of the knottin (or other protein to be engineered) is fused to Aga2, but several alternative variations of the yeast surface display plasmid have been described and may be employed to develop a knottin peptide for use in a bifunctional molecule of the present disclosure. One of the benefits of this screening platform over panning-based methods used with phage or mRNA display is that two-color FACS can be used to quantitatively discriminate clones that differ by as little as two-fold in binding affinity to the desired target.

To selectively mutate knottin loop regions at the DNA level, degenerate codons can be introduced by oligonucleotide assembly using, e.g., overlap extension PCR. Next, the genetic material may be amplified using flanking primers with sufficient overlap with the yeast display vector for homologous recombination in yeast. This assembly and amplification method allow knottin libraries to be created at relatively low cost and effort. Synthetic oligonucleotide libraries and recent methods have been developed that allow defined control over library composition.

In certain aspects, a display library (e.g., a yeast display library) is screened for binding to the cell surface or extracellular molecule of interest by FACS. When screening knottin libraries by FACS, an enriched pool of binders generally emerges in 4-7 rounds of sorting. Two-color FACS may be used for library screening, where one fluorescent label can be used to detect the c-myc epitope tag and the other to measure interaction of the knottin mutant against the binding target of interest. Different instrument lasers and/or filter sets can be used to measure excitation and emission properties of the two fluorophores at single-cell resolution. This enables yeast expression levels to be normalized with binding. That is, a knottin that exhibits poor yeast expression but binds a high amount of a target can be distinguished from a knottin that is expressed at high levels but binds weakly to a target. Accordingly, a two-dimensional flow cytometry plot of expression versus binding will result in a diagonal population of yeast cells that bind to target antigen. High-affinity binders can be isolated using library sort gates. Alternatively, in an initial sort round it could be useful to clear the library of undesired clones that do not express full-length.

The target used in the screening is structurally and functionally relevant for the final application, e.g., mimics the cell surface or extracellular molecule of interest. In certain aspects, the target used in the screening is HER2, CD19, CD22, CD30, CD33, CD56, CD66/CEACAM5, CD70, CD74, CD79b, CD138, Nectin-4, Mesothelin, Transmembrane glycoprotein NMB (GPNMB), Prostate-Specific Membrane Antigen (PSMA), SLC44A4, CA6, CA-IX, αvβ1 integrin, αvβ3 integrin, αvβ5 integrin, αvβ6 integrin, α5β1 integrin, C—X—C chemokine receptor type 4 (CXCR4), cytotoxic T-lymphocyte-associated protein 4 (CTLA-4), neuropilin-1 (NRP1), matriptase, or any other tumor-associated or tumor-specific cell surface molecules of interest.

Following enrichment of knottin libraries for binders against the cell surface or extracellular molecule of interest, the yeast plasmids are recovered and sequenced. Additional rounds of FACS can be performed under increased sorting stringency. The binding affinities or kinetic off-rates of individual yeast-displayed knottin clones may then be measured.

Once knottin peptides having an engineered loop that binds to the cell surface or extracellular molecule of interest have been identified by surface display (e.g., yeast surface display), the engineered knottins may be produced using a suitable method. The small size of knottins makes them amenable to production by both chemical synthesis and recombinant expression. According to certain embodiments, the knottin peptide may be produced by solid phase peptide synthesis followed by in vitro folding. Chemical synthesis permits facile incorporation of unnatural amino acids or other chemical handles into knottin peptides.

Knottin peptide sequences are readily synthesized using solid phase peptide chemistry on an automated synthesizer. For example, standard 9-fluorenylmethyloxycarbonyl (Fmoc)-based solid phase peptide chemistry may be employed. The linear peptide may then be folded under conditions that promote oxidation of cysteine side chain thiols to form disulfide bonds, followed by purification, e.g., by reversed-phase high-performance liquid chromatography (RP-HPLC).

In certain aspects, the knottin protein is produced using a recombinant DNA approach. Strategies have been developed for producing knottins using recombinant methods in a variety of host cell types. For example, functional knottins have been produced with barnase as a genetic fusion partner, which promotes folding in the E. coli periplasmic space and serves as a useful purification handle. According to certain embodiments, the engineered knottin peptide is expressed in yeast. The yeast strain Pichia pastoris, for example, has been successfully employed to produce 2-10 mg/L of purified engineered knottins. The yeast expression construct may encode one or more tags (e.g., a C-terminal hexahistadine tag for purification by, e.g., metal chelating chromatography (Ni-NTA)). Size exclusion chromatography may then be used to remove aggregates, misfolded multimers, and the like.

Aspects of the present disclosure include nucleic acids that encode the knottin peptides employed in the bifunctional molecules of the present disclosure. That is, provided are nucleic acids that encode any of the subject knottin peptides described herein having an engineered loop that binds to a cell surface or extracellular molecule of interest. In certain aspects, such a nucleic acid is present in an expression vector. The expression vector includes a promoter operably linked to the nucleic acid encoding the knottin peptide, the promoter being selected based on the type of host cell selected to express the knottin peptide. Also provided are host cells that include any of the knottin peptide-encoding nucleic acids of the present disclosure, as well as any expression vectors including the same.

Methods are available for measuring the affinity of knottins for molecules expressed on the surface of cells (e.g., cancer cells, such as mammalian cancer cells) using direct binding or competition binding assays. In a direct binding assay, an equilibrium binding constant (K_{D}) may be measured using a knottin conjugated to a fluorophore or radioisotope, or a knottin that contains an N- or C-terminal epitope tag for detection by a labeled antibody. If labels or tags are not feasible or desired, a competition binding assay can be used to determine the half-maximal inhibitory concentration (IC₅₀), the amount of unlabeled knottin at which 50% of the maximal signal of the labeled competitor is detectable. A K_{D} value can then be calculated from the measured IC₅₀ value. Ligand depletion will be more pronounced when measuring high-affinity interactions over a lower concentration range, and can be avoided or minimized by decreasing the number of cells added in the experiment or by increasing the binding reaction volumes.

In certain aspects, the knottin peptide has an equilibrium binding constant (K_{D}) for the cell-surface molecule of from about 0.01 nM to 100 nM, such as from about 0.025 nM to 75 nM, about 0.05 nM to 50 nm, about 0.075 nM to 25 nM, or from about 0.1 nM to 10 nM. In some embodiments, the knottin peptide has an equilibrium binding constant (K_{D}) for the cell-surface molecule of from about 0.1 nM to 10 nM. In some embodiments, the knottin peptide has an equilibrium binding constant (K_{D}) for the cell-surface molecule of about 0.1 nM. In some embodiments, the knottin peptide has an equilibrium binding constant (K_{D}) for the cell-surface molecule of about 0.5 nM. In some embodiments, the knottin peptide has an equilibrium binding constant (K_{D}) for the cell-surface molecule of about 1 nM. In some embodiments, the knottin peptide has an equilibrium binding constant (K_{D}) for the cell-surface molecule of about 5 nM. In some embodiments, the knottin peptide has an equilibrium binding constant (K_{D}) for the cell-surface molecule of about 10 nM.

Detailed guidance and specific protocols for engineering knottins by yeast surface display technology, including knottin library construction and screening, as well as knottin production by chemical synthesis and recombinant expression, and further for cell binding assays to measure the affinity of knottins to cell surface and extracellular molecules, are described in Moore, S. and Cochran, J. (2012) Engineering Knottins as Novel Binding Agents, Methods in Enzymology, 503, 223-251.

Integrin-Binding First Moieties

In some embodiments, the first moiety is an integrin-binding moiety. Such a moiety may be any type of moiety capable of binding to an integrin to be targeted for degradation via the endosomal/lysosomal pathway. In certain aspects, such a first moiety is selected from a polypeptide, a ligand (e.g., a ligand for the integrin, where the integrin is targeted for degradation), an aptamer, a nanoparticle, and a small molecule. The second moiety may be any type moiety capable of binding to the lysosomal targeting molecule. In certain embodiments, the second moiety is selected from a polypeptide, a ligand (e.g., a ligand for the lysosomal targeting molecule), an aptamer, a nanoparticle, and a small molecule.

In some embodiments, when the integrin-binding first moiety is a polypeptide, the moiety is an antibody. The terms “antibody” and “immunoglobulin” include antibodies or immunoglobulins of any isotype (e.g., IgG (e.g., IgG1, IgG2, IgG3 or IgG4), IgE, IgD, IgA, IgM, etc.), whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the cell surface molecule or extracellular molecule (in the case of the first moiety) or lysosomal targeting molecule (in the case of the second moiety), including, but not limited to, Fv, single chain Fv (scFv), Fab, F(ab′)₂, Fab′, (scFv′)₂, diabodies, and nanobodies; chimeric antibodies; monoclonal antibodies; fully human antibodies; humanized antibodies (e.g., humanized whole antibodies, humanized antibody fragments, etc.); and fusion proteins including an antigen-binding portion of an antibody and a non-antibody protein or fragment thereof. The antibodies may be detectably labeled, e.g., with an in vivo imaging agent, or the like. The antibodies may be further conjugated to other moieties, such as, e.g., polyethylene glycol (PEG), etc. Fusion to an antibody Fc region (or a fragment thereof), conjugation to PEG, etc. may find use, e.g., for increasing serum half-life of the antibody upon administration to the subject.

Non-limiting examples of integrin-binding antibodies that may be employed in the bifunctional molecules of the present disclosure include, e.g., etrolizumab, odulimomab, efalizumab, erlizumab, rovelizumab, abrilumab, vedolizumab, natalizumab, SAN-300, vatelizumab, P5, efatucizumab, STX-200, STX-100, volociximab, and many others, including integrin-binding fragments and variants thereof.

According to certain embodiments, an integrin-binding first moiety is a ligand for one or more integrins. As used herein, a “ligand” is a substance that forms a complex with a biomolecule to serve a biological purpose. The ligand may be a substance that forms a complex with the integrin(s) on the surface of the target cell. In certain embodiments, when the integrin-binding first moiety is a ligand, the ligand is modified in such a way that complex formation with the integrin occurs, but the normal biological result of such complex formation does not occur. In certain embodiments, when the integrin-binding first moiety is a ligand for one or more integrins, the ligand comprises an amino acid sequence (e.g., an amino acid sequence comprising the sequence RGD) which is recognized and bound by the one or more integrins. Non-limiting examples of integrin ligands that may be employed in the bifunctional molecules of the present disclosure include, e.g., any of the integrin binding knottin peptides described herein (e.g., EETI-2.5F, EETI-2.5D, integrin binding variants thereof, etc.), echistatin, RGD, RGDS, GRGD, GRGDS, GRGDSP, GRGDSPK, GRGDNP, GRGDTP, c(RGDfV), cilengitide, c(RGDfK), c(RGDyK), c(RGDfC), and many others.

In certain embodiments, the integrin-binding first moiety is an aptamer. By “aptamer” is meant a nucleic acid (e.g., an oligonucleotide) that has a specific binding affinity for one or more integrins. Aptamers exhibit certain desirable properties, such as ease of selection and synthesis, high binding affinity and specificity, low immunogenicity, and versatile synthetic accessibility. Aptamers that bind to cell surface molecules are known and include, e.g., TTA1 (a tumor targeting aptamer to the extracellular matrix protein tenascin-C).

According to certain embodiments, an integrin-binding first moiety is a small molecule. By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain embodiments, the small molecule is not made of repeating molecular units such as are present in a polymer. Non-limiting examples of integrin-binding small molecules that may be employed in the bifunctional molecules of the present disclosure include, e.g., eptifibatide, tirofiban, Tc-99m-P280, SF-0166, cilengitide, GSK3008348, E-7820, lifitegrast, and many others.

Lysosomal Targeting Molecules

As summarized above, the second moiety specifically binds a lysosomal targeting molecule. As used herein, a “lysosomal targeting molecule” is a cell surface molecule which, upon being bound by the second moiety of the bifunctional molecule, shuttles the bifunctional molecule and cell surface molecule or extracellular molecule bound by the first moiety to the lysosome within the cell. Upon delivery and internalization into the lysosome, the bifunctional molecule and cell surface molecule or extracellular molecule are degraded by lysosomal enzymes, e.g., acid hydrolases. In this way, the bifunctional molecule targets the cell surface molecule or extracellular molecule bound by the first moiety for degradation, which targeting finds use in a variety of in vitro and in vivo applications, including research and clinical applications.

The second moiety may bind to any suitable lysosomal targeting molecule. Non-limiting examples of lysosomal targeting molecules include a mannose-6-phosphate receptor (M6PR), sortilin, folate receptor, ASGPR, IFITM3, molecules in the endosome/lysosome pathway (e.g., LIMP-1, LIMP-2), etc.

In some embodiments, the lysosomal targeting molecule to which the second moiety binds is a mannose-6-phosphate receptor (M6PR). M6PRs are present throughout the tissues of the body and are responsible for trafficking mannose-6-phosphate (M6P)-tagged cargo, such as acid hydrolases, from the Golgi compartment and extracellular space to the lysosome. Details regarding M6PRs may be found, e.g., in Gary-Bobo et al. (2007) Curr. Med. Chem. 14:2945-2953; Das et al. (2016) ACS Macro Lett. 5:809-813; and elsewhere. Examples of M6PRs to which the second moiety may bind include the cation-dependent human M6PR provided as UniProtKB-P20645 and the cation-independent M6PR provided as UniProtKB-P11717 (also referred to as insulin-like growth factor 2 receptor (IGF2R)). The cation-independent mannose 6-phosphate receptor is a multifunctional protein which binds at the cell surface to ligands such as mannose 6-phosphate (M6P) bearing proteins, IGF-II, retinoic acid, plasminogen, etc. Its major function is to bind and transport M6P-enzymes to lysosomes, but it can also modulate the activity of a variety of extracellular M6P-glycoproteins, e.g., latent TGFβ precursor, urokinase-type plasminogen activator receptor, granzyme B, growth factors, herpes virus, etc.

In certain aspects, when the lysosomal targeting molecule is an M6PR, the second moiety is an antibody that specifically binds the M6PR. Anti-M6PR antibodies are available and include the MOB-1772z recombinant anti-human M6PR antibody (Creative Biolabs), the EPR6599, 2G11, MEM-238, EPR6599, and EPR7691 anti-M6PR antibodies (Abcam), and the like.

In some embodiments, when the lysosomal targeting molecule is an M6PR, the second moiety includes one or more M6PR ligands. In certain aspects, the one or more M6PR ligands include one or more mannose-6-phosphates (M6Ps), where M6P has the following structure:

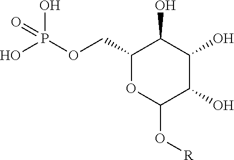

Alternatively, or additionally, the one or more M6PR ligands include one or more M6P analogs. By “M6P analog” is meant a molecule that is not M6P but binds to an M6P recognition site of M6PR. Several M6P analogs with phosphonate, carboxylate, sulfate, sulfonate or malonate groups display a higher affinity and a stronger stability in human serum than M6P itself. Some structural features have been shown to be important for the binding of M6P to M6PR. For example, the hydroxyl group at the 2-position of the pyranose ring is axial, strong binding to M6PR is observed. The distance between the negative charge and the pyranose ring also plays a role in M6P recognition by M6PR. It has been shown that suitable analogs: should generally be isosteric to M6P to efficiently bind M6PR; a single negative charge is sufficient to allow binding to M6PR while the phosphorus atom is not necessary to ensure recognition; and the presence of two negative charges (as in the malonate and phosphonate isosteric analogs of M6P) is beneficial for binding to M6PR. In some embodiments, when the one or more M6PR ligands include one or more M6PR analogs, the one or more M6PR analogs include one or more phosphonate M6P analogs (M6Pn), malonate M6P analogs, carboxylate M6P analogs, sulfonate M6P analogs, acrylate M6P analogs, and/or the like. In some embodiments, the one or more M6PR analogs include one or more phosphonate M6P analogs (M6Pn) having the structure (where M⁺ is any countercation or hydrogen atom):

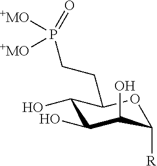

In some embodiments, the one or more M6PR analogs include one or more carboxylate M6P analogs having one of the following structures (where M⁺ is any countercation or hydrogen atom):

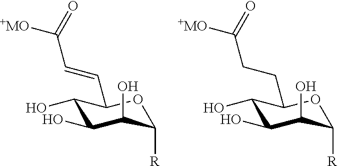

In some embodiments, the one or more M6PR analogs include one or more malonate M6P analogs having the following structure (where M⁺ is any countercation or hydrogen atom):

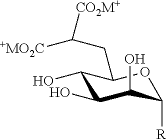

Details regarding M6P and M6P analog recognition by M6PR, as well as M6P analogs that may be employed in the bifunctional molecules of the present disclosure may be found, e.g., in Gary-Bobo et al. (2007) Curr. Med. Chem. 14:2945-2953; and Jeanjean et al. (2008) Bioorg Med Chem Lett. 18(23): 6240-3, the disclosures of which are incorporated herein by reference in their entireties for all purposes.

In certain aspects, when the second moiety includes one or more M6PR ligands, the second moiety includes from 1 to 1000 M6PR ligands, such as from 1 to 750, 1 to 500, 1 to 250, 1 to 100, 1 to 75, 1 to 50, 1 to 40, 1 to 30, 1 to 20, 1 to 10 (e.g., 1 to 6), or 1 to 5 M6PR ligands. In some embodiments, when the second moiety includes one or more M6PR ligands, the second moiety includes from 10 to 50, 15 to 45, 20 to 40, or 25 to 35 M6PR ligands. In certain aspects, when the second moiety includes one or more M6PR ligands, the second moiety includes 5 or more, 10 or more, 20 or more, 30 or more, 40 or more, 50 or more, 75 or more, 100 or more, 250 or more, 500 or more, 750 or more, or 1000 or more M6PR ligands.

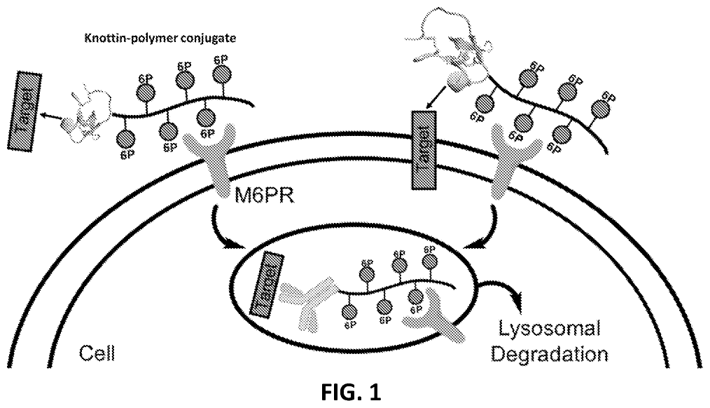

In some embodiments, when the second moiety includes one or more M6PR ligands, the second moiety includes a polymer scaffold that displays (e.g., is functionalized with) the one or more M6PR ligands. One example of such a bifunctional molecule and use thereof is schematically illustrated in FIG. 1. In this example, the bifunctional molecule includes a knottin peptide as the first moiety. The knottin peptide may bind either a target extracellular molecule (as shown on the left) or a target cell surface molecule (as shown on the right). The knottin peptide is conjugated to a polymer scaffold that displays M6P ligands (each of which is designated “6P” in FIG. 1). Upon binding of a displayed M6P ligand by a cell surface M6PR, the M6PR shuttles the bifunctional molecule (and bound target molecule) to the lysosome for degradation.

When the second moiety includes a polymer scaffold that displays the one or more M6PR ligands, the polymer scaffold may be a glycopolymer including the one or more M6PR ligands. By way of example, the glycopolymer may be a glycoprotein including one or more amino acids (e.g., natural and/or non-natural amino acids) functionalized with the one or more M6PR ligands. When the glycopolymer is a glycoprotein, the glycoprotein may be a N-carboxyanhydride (NCA)-derived glycoprotein. The ring-opening polymerization (ROP) of NCA monomers is a well-studied route to synthetic polypeptides and polypeptide hybrids that possess a broad range of useful physical properties. In some embodiments, the polymerization is metal-catalyzed. Suitable approaches for large-scale synthesis of α-amino acid-N-carboxyanhydrides are described, e.g., in Semple et al. (2016) Synthetic Communications 47(1):53-61.

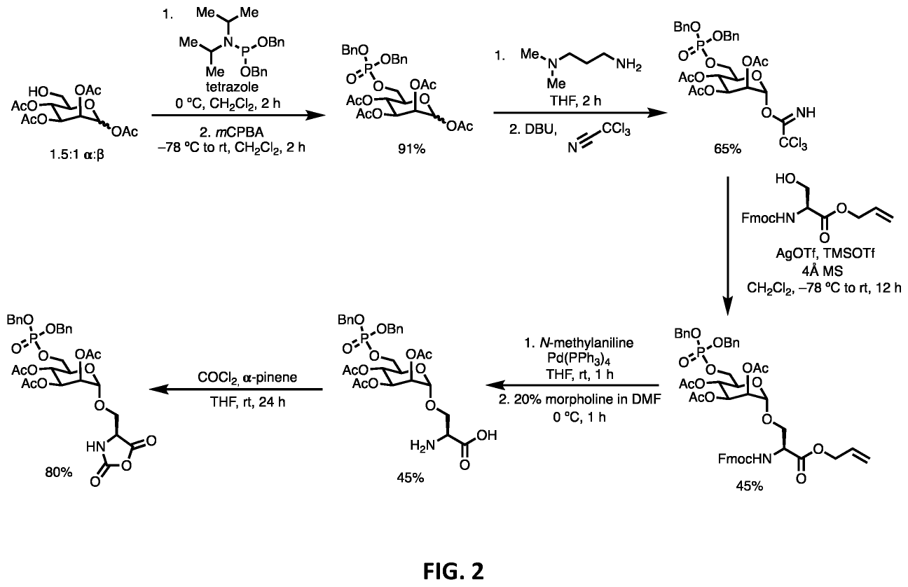

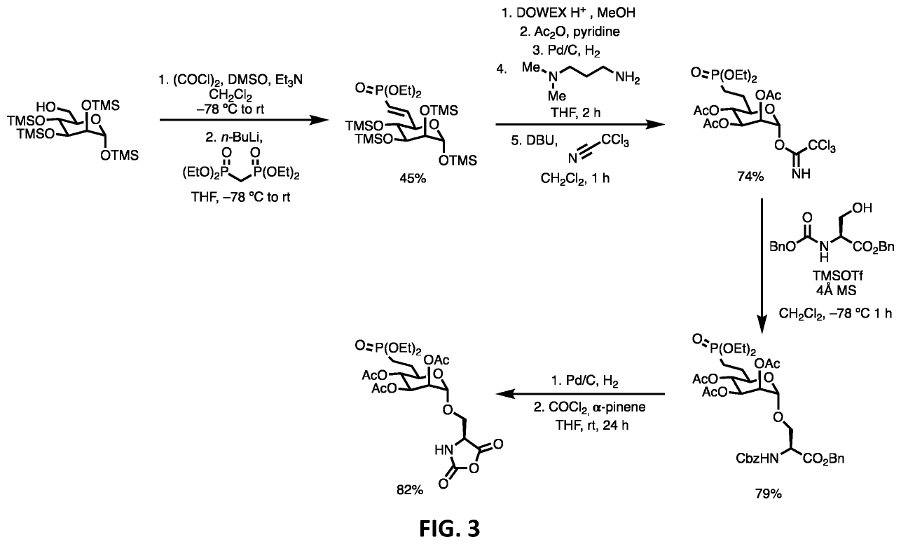

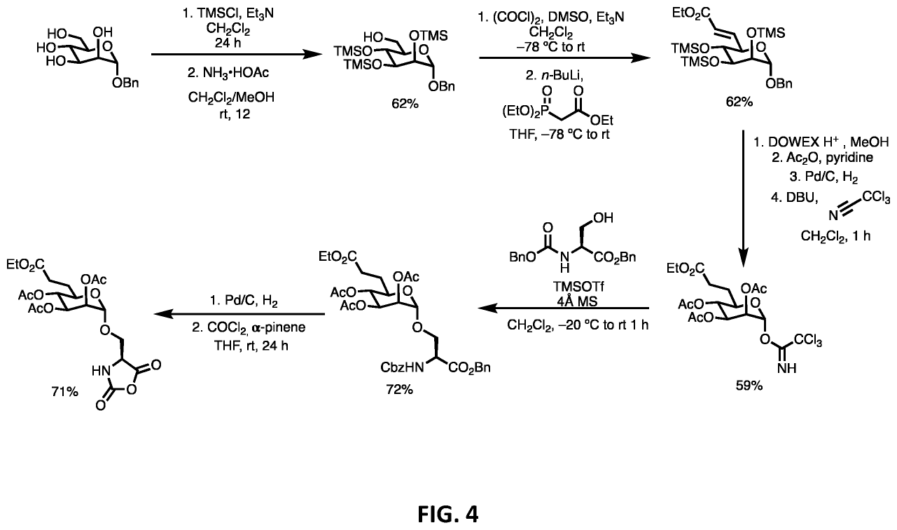

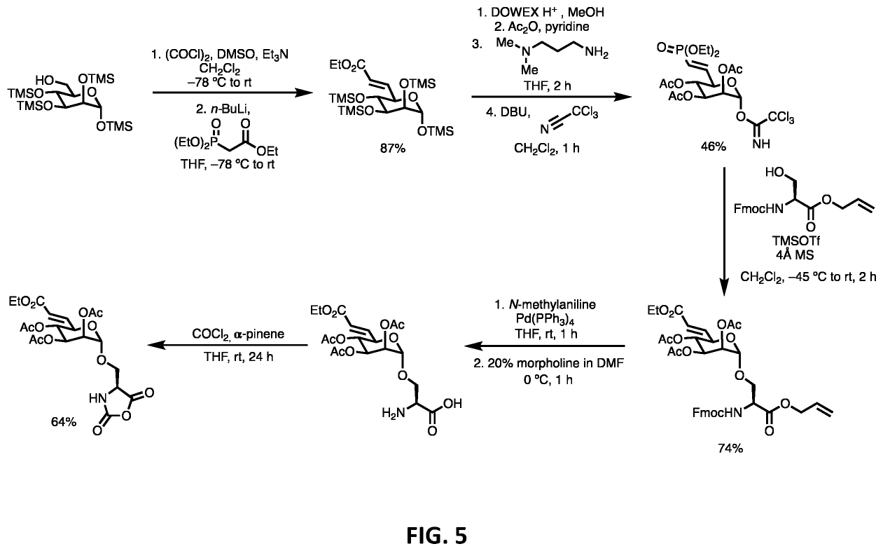

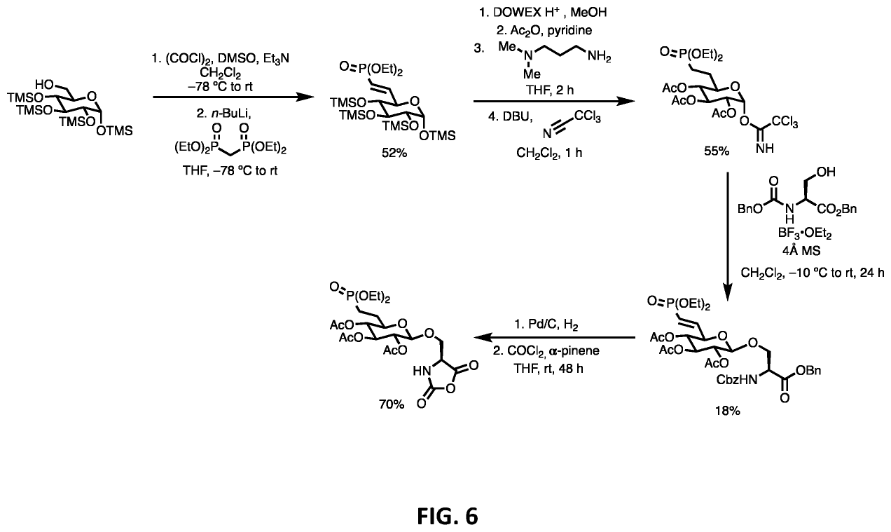

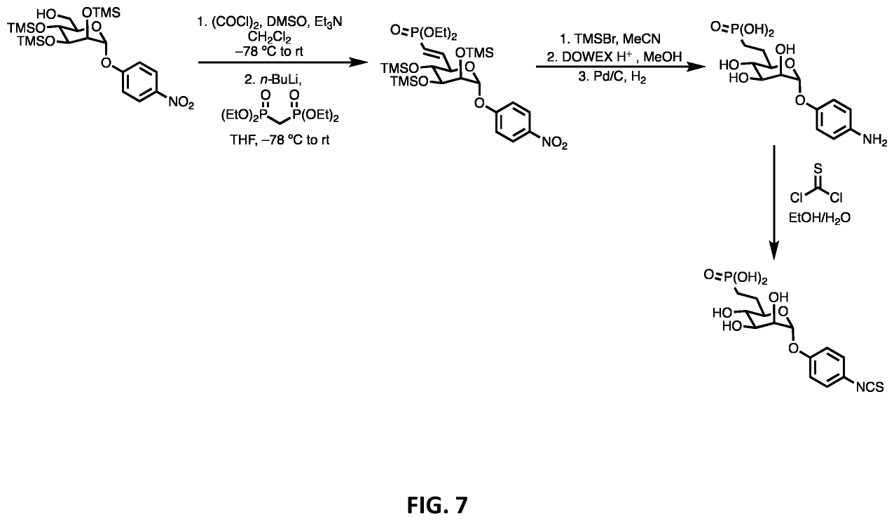

An example approach for synthesizing mannose-6-phosphate N-carboxyanhydride is schematically illustrated in FIG. 2. An example approach for synthesizing mannose-6-phosphonate N-carboxyanhydride is schematically illustrated in FIG. 3. An example approach for synthesizing mannose-6-carboxylate N-carboxyanhydride is schematically illustrated in FIG. 4. An example approach for synthesizing mannose-6-acrylate N-carboxyanhydride is schematically illustrated in FIG. 5. An example approach for synthesizing glucose-6-phosphonate N-carboxyanhydride is schematically illustrated in FIG. 6. An example approach for synthesizing mannose-6-phosphonate isothiocyanate is schematically illustrated in FIG. 7.

In certain embodiments, a bifunctional molecule of the present disclosure comprises a second moiety that specifically binds a lysosomal targeting molecule expressed on the surface of liver cells, e.g., hepatocytes (including hepatocellular carcinoma (HCC) cells). A non-limiting example of a lysosomal targeting molecule expressed on the surface of liver cells to which the second moiety may bind is asialoglycoprotein receptor (ASGPR)—a hepatic receptor that mediates removal of glycoconjugates from blood. The receptor comprises two proteins, asialoglycoprotein receptor 1 and 2 (ASGR1 (UniProtKB-P07306 human) and ASGR2 (UniProtKB-P07307-human)), encoded by the genes ASGR1 and ASGR2. ASGPR binds asialoglycoproteins, which are glycoproteins from which a sialic acid has been removed to expose galactose and galactosamine residues. The receptors, which are located on liver cells, remove the target glycoproteins from circulation. ASGPR is highly expressed on the surface of hepatocytes, several human carcinoma cell lines, and liver cancers.

When the lysosomal targeting molecule is ASGPR, suitable second moieties include but are not limited to anti-ASGPR antibodies, ASGPR ligands, and the like. According to some embodiments, such a second moiety comprises one or more ASGPR ligands. Suitable ASGPR ligands include, but are not limited to, one or more N-acetylgalactosamines (GaINAc), one or more galactoses, one or more glucoses, and any combination thereof. In certain embodiments, such a second moiety comprises from 1 to 1000 ASGPR ligands, such as from 1 to 750, 1 to 500, 1 to 250, 1 to 100, 1 to 75, 1 to 50, 1 to 40, 1 to 30, 1 to 20, 1 to 10 (e.g., 1 to 6), or 1 to 5 ASGPR ligands. In some embodiments, when the second moiety includes one or more ASGPR ligands, the second moiety includes from 10 to 50, 15 to 45, 20 to 40, or 25 to 35 ASGPR ligands. In certain embodiments, when the second moiety includes one or more ASGPR ligands, the second moiety includes 3 or more, 5 or more, 10 or more, 20 or more, 30 or more, 40 or more, 50 or more, 75 or more, 100 or more, 250 or more, 500 or more, 750 or more, or 1000 or more ASGPR ligands.

According to some embodiments, when the lysosomal targeting molecule is ASGPR and the second moiety comprises one or more ASGPR ligands, the second moiety comprises a scaffold comprising the one or more ASGPR ligands. In one non-limiting example, the second moiety comprises a polymer comprising GaINAc. In certain embodiments, such a second moiety comprises poly(GaINAc-co-Ala), the structure of which is provided below and in FIG. 20.

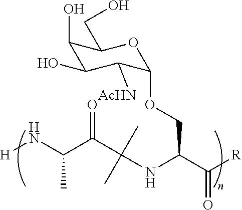

In certain embodiments, when the lysosomal targeting molecule is ASGPR and the second moiety comprises one or more ASGPR ligands, the second moiety comprises a dendrimer scaffold comprising 1 (monovalent), 2 (bivalent), 3 (trivalent) or 4 or more ASGPR ligands, e.g., ASGPR ligands independently selected from GaINAc, galactose, and glucose. For example, according to some embodiments, the second moiety comprises a monovalent, bivalent, or trivalent GaINAc-containing dendrimer scaffold. A non-limiting example of a trivalent GaINAc-containing dendrimer scaffold that may be employed is the following (designated herein as Tri-GaINAc dendrimer):

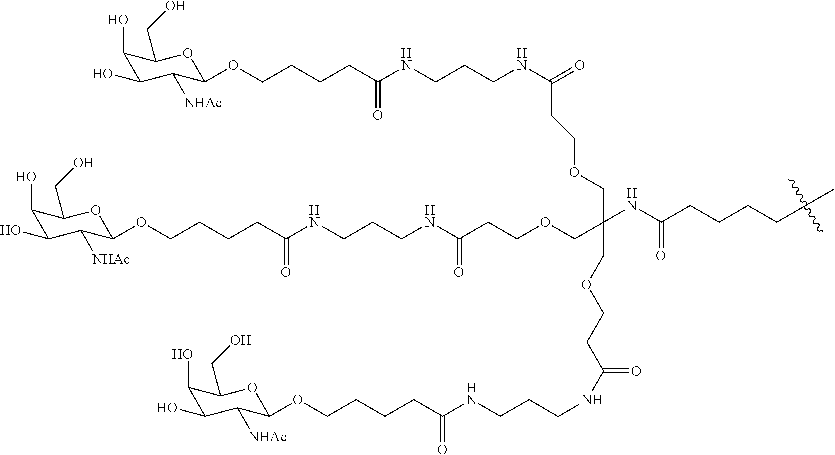 [SMILES: *CCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

According to some embodiments, the second moiety comprises a monovalent, bivalent, or trivalent galactose-containing dendrimer scaffold. A non-limiting example of a trivalent galactose-containing dendrimer scaffold that may be employed is the following (designated herein as Tri-Gal dendrimer):

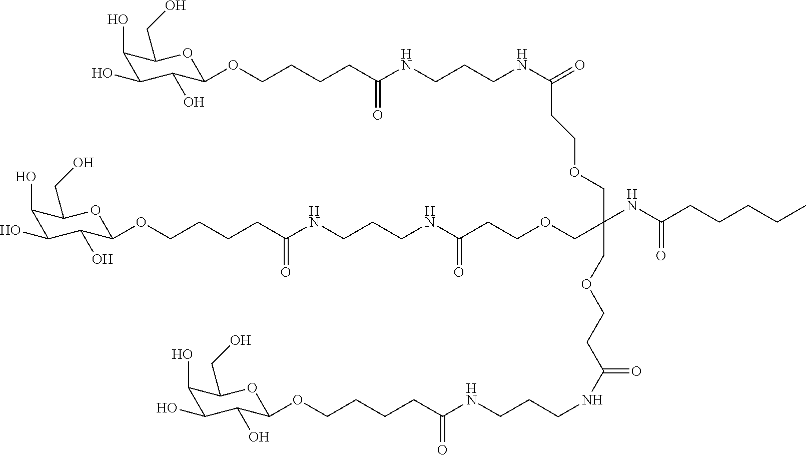 [SMILES: CCCCCC(=O)NC(COCCC(=O)NCCCNC(=O)CCCCOC1OC(CO)C(O)C(O)C1O)(COCCC(=O)NCCCNC(=O)CCCCOC1OC(CO)C(O)C(O)C1O)COCCC(=O)NCCCNC(=O)CCCCOC1OC(CO)C(O)C(O)C1O]

Cell Surface Molecules and Extracellular Molecules

In some embodiments, the bifunctional molecules of the present disclosure comprise a first moiety (e.g., a knottin peptide) that specifically binds a cell surface molecule or extracellular molecule. In some embodiments, the first moiety specifically binds a cell surface molecule. By “cell surface molecule” is meant a molecule associated with a cell membrane, e.g., because the molecule has a domain that inserts into or spans a cell membrane, e.g., a cell membrane-tethering domain or a transmembrane domain. The cell surface molecule may be any cell surface molecule which is desired for targeted degradation via the endosomal/lysosomal pathway. In some embodiments, the cell surface molecule is a cell surface receptor. Cell surface receptors of interest include, but are not limited to, cell adhesion receptors, stem cell receptors, immune cell receptors, growth factor receptors, cytokine receptors, hormone receptors, receptor tyrosine kinases, a receptor in the epidermal growth factor receptor (EGFR) family (e.g., HER2 (human epidermal growth factor receptor 2), etc.), a receptor in the fibroblast growth factor receptor (FGFR) family, a receptor in the vascular endothelial growth factor receptor (VEGFR) family, a receptor in the platelet derived growth factor receptor (PDGFR) family, a receptor in the rearranged during transfection (RET) receptor family, a receptor in the Eph receptor family, a receptor in the discoidin domain receptor (DDR) family, and a mucin protein (e.g., MUC1). In some embodiments, the cell surface molecule is CD71 (transferrin receptor). In certain embodiments, the cell surface receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

In some embodiments, the first moiety specifically binds a cell surface molecule which mediates its effect not through a specific molecular interaction (and therefore is not susceptible to blocking), but rather through bulk biophysical or aggregate effects. A non-limiting example of such a cell surface molecule is a mucin. Examples of mucins include, but are not limited to, MUC1, MUC16, MUC2, MUC5AC, MUC4, CD43, CD45, GPIb, and the like.

According to some embodiments, the first moiety (e.g., a knottin peptide) specifically binds one or more cell adhesion receptors. In certain aspects, when the cell surface molecule is a cell adhesion receptor, the cell adhesion receptor is an integrin. For example, a bifunctional molecule of the present disclosure may comprise a knottin peptide having a loop engineered to bind to any one of αvβ1 integrin, αvβ3 integrin, αvβ5 integrin, αvβ6 integrin, α5β1 integrin, or any combination thereof. According to certain embodiments, the engineered loop binds to each of αvβ1 integrin, αvβ3 integrin, αvβ5 integrin, αvβ6 integrin, and α5β1 integrin.

An EETI-based knottin peptide (designated EETI-2.5D) having an engineered binding loop that binds to each of αvβ1 integrin, αvβ3 integrin, αvβ5 integrin, αvβ6 integrin, and α5β1 integrin, which may be employed in a bifunctional molecule of the present disclosure, has the following amino acid sequence (with the integrin-binding loop underlined):

(SEQ ID NO: 7)
GCPQGRGDWAPTSCKQDSDCRAGCVCGPNGFCG

An EETI-based knottin peptide (designated EETI-2.5F) having an engineered binding loop that binds to each of αvβ1 integrin, αvβ3 integrin, αvβ5 integrin, αvβ6 integrin, and α5β1 integrin, which may be employed in a bifunctional molecule of the present disclosure, has the following amino acid sequence (with the integrin-binding loop underlined):

(SEQ ID NO: 8)
GCPRPRGDNPPLTCKQDSDCLAGCVCGPNGXCG,

where X=F or Y.

In some embodiments, the knottin peptide of a bifunctional molecule of the present disclosure is an integrin-binding knottin peptide comprising an amino acid sequence having 70% or greater, 75% or greater, 80% or greater, 85% or greater, 90% or greater, 95% or greater, or 100% identity to the amino acid sequence of an EETI-based knottin peptide as set forth in Table 1.

TABLE 1
Example EETI Integrin-Binding Knottin Peptides

| Peptide | | |
|---|---|---|
| identifier | Sequence | SEQ ID NO: |
| 1.4A | GCAEPRGDMPWTWCKQDSDCLAGCVCGPNGFCG | (SEQ ID NO: 9) |
| 1.4B | GCVGGRGDWSPKWCKQDSDCPAGCVCGPNGFCG | (SEQ ID NO: 10) |
| 1.4C | GCAELRGDRSYPECKQDSDCLAGCVCGPNGFCG | (SEQ ID NO: 11) |
| 1.4E | GCRLPRGDVPRPHCKQDSDCQAGCVCGPNGFCG | (SEQ ID NO: 12) |
| 1.4H | GCYPLRGDNPYAACKQDSDCRAGCVCGPNGFCG | (SEQ ID NO: 13) |
| 1.5B | GCTIGRGDWAPSECKQDSDCLAGCVCGPNGFCG | (SEQ ID NO: 14) |
| 1.5F | GCHPPRGDNPPVTCKQDSDCLAGCVCGPNGFCG | (SEQ ID NO: 15) |
| 2.3A | GCPEPRGDNPPPSCKQDSDCRAGCVCGPNGFCG | (SEQ ID NO: 16) |
| 2.3B | GCLPPRGDNPPPSCKQDSDCQAGCVCGPNGFCG | (SEQ ID NO: 17) |
| 2.3C | GCHLGRGDWAPVGCKQDSDCPAGCVCGPNGFCG | (SEQ ID NO: 18) |
| 2.3D | GCNVGRGDWAPSECKQDSDCPAGCVCGPNGFCG | (SEQ ID NO: 19) |
| 2.3E | GCFPGRGDWAPSSCKQDSDCRAGCVCGPNGFCG | (SEQ ID NO: 20) |
| 2.3F | GCPLPRGDNPPTECKQDSDCQAGCVCGPNGFCG | (SEQ ID NO: 21) |
| 2.3G | GCSEARGDNPRLSCKQDSDCRAGCVCGPNGFCG | (SEQ ID NO: 22) |
| 2.3H | GCLLGRGDWAPEACKQDSDCRAGCVCGPNGFCG | (SEQ ID NO: 23) |
| 2.3I | GCHVGRGDWAPLKCKQDSDCQAGCVCGPNGFCG | (SEQ ID NO: 24) |
| 2.3J | GCVRGRGDWAPPSCKQDSDCPAGCVCGPNGFCG | (SEQ ID NO: 25) |
| 2.4A | GCLGGRGDWAPPACKQDSDCRAGCVCGPNGFCG | (SEQ ID NO: 26) |
| 2.4C | GCFVGRGDWAPLTCKQDSDCQAGCVCGPNGFCG | (SEQ ID NO: 27) |
| 2.4D | GCPVGRGDWSPASCKQDSDCRAGCVCGPNGFCG | (SEQ ID NO: 28) |
| 2.4E | GCPRPRGDNPPLTCKQDSDCLAGCVCGPNGFCG | (SEQ ID NO: 29) |
| 2.4F | GCYQGRGDWSPSSCKQDSDCPAGCVCGPNGFCG | (SEQ ID NO: 30) |
| 2.4G | GCAPGRGDWAPSECKQDSDCQAGCVCGPNGFCG | (SEQ ID NO: 31) |
| 2.4J | GCVQGRGDWSPPSCKQDSDCPAGCVCGPNGFCG | (SEQ ID NO: 32) |
| 2.5A | GCHVGRGDWAPEECKQDSDCQAGCVCGPNGFCG | (SEQ ID NO: 33) |
| 2.5C | GCDGGRGDWAPPACKQDSDCRAGCVCGPNGFCG | (SEQ ID NO: 34) |
| 2.5D | GCPQGRGDWAPTSCKQDSDCRAGCVCGPNGFCG | (SEQ ID NO: 7) |
| 2.5F | GCPRPRGDNPPLTCKQDSDCLAGCVCGPNGXCG | (SEQ ID NO: 8) |
| 2.5H | GCPQGRGDWAPEWCKQDSDCPAGCVCGPNGFCG | (SEQ ID NO: 35) |
| 2.5J | GCPRGRGDWSPPACKQDSDCQAGCVCGPNGFCG | (SEQ ID NO: 36) |

In some embodiments, the knottin peptide of a bifunctional molecule of the present disclosure is an integrin-binding knottin peptide comprising an amino acid sequence having 70% or greater, 75% or greater, 80% or greater, 85% or greater, 90% or greater, 95% or greater, or 100% identity to the amino acid sequence of an AgRP-based knottin peptide as set forth in Table 2.

TABLE 2
Example AgRP Integrin-Binding Knottin Peptides

| Clone | Loop 4 sequence |
|---|---|
| 7A (5E) (SEQ ID NO: 37) | GCVRLHESCLGQQVPCCDPAATCYCSGRGDNDLVCYCR |
| 7B (SEQ ID NO: 38) | GCVRLHESCLGQQVPCCDPAATCYCKGRGDARLQCYCR |
| 7E (SEQ ID NO: 39) | GCVRLHESCLGQQVPCCDPAATCYCVGRGDDNLKCYCR |
| 7J (6B) (SEQ ID NO: 40) | GCVRLHESCLGQQVPCCDPAATCYCEGRGDRDMKCYCR |
| 7C (SEQ ID NO: 41) | GCVRLHESCLGQQVPCCDPAATCYCYGRGDNDLRCYCR |

In some embodiments, when the first moiety is a knottin peptide that specifically binds a cell surface molecule, the cell surface molecule is present on a cancer cell. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like. In some embodiments, the cell surface molecule present on the cancer cell is a tumor-associated antigen or a tumor-specific antigen.

In certain embodiments, when the first moiety is a knottin peptide that specifically binds a cell surface molecule, the cell surface molecule is present on an immune cell. In some embodiments, the cell surface molecule is present on an immune cell selected from a T cell, a B cell, a natural killer (NK) cell, a macrophage, a monocyte, a neutrophil, a dendritic cell, a mast cell, a basophil, and an eosinophil. In certain aspects, the cell surface molecule present on the immune cell is an inhibitory immune receptor. As used herein, an “inhibitory immune receptor” is a receptor present on an immune cell that negatively regulates an immune response. Examples of inhibitory immune receptors which may be inhibited according to the methods of the present disclosure include inhibitory immune receptors of the Ig superfamily, including but not limited to: CD200R, CD300a (IRp60; mouse MAIR-1), CD300f (IREM-1), CEACAM1 (CD66a), FcγRlIb, ILT-2 (LIR-1; LILRB1; CD85j), ILT-3 (LIR-5; CD85k; LILRB4), ILT-4 (LIR-2; LILRB2), ILT-5 (LIR-3; LILRB3; mouse PIR-B); LAIR-1, PECAM-1 (CD31), PILR-α (FDF03), SIRL-1, and SIRP-α. Further examples of inhibitory immune receptors which may be inhibited according to the methods of the present disclosure include sialic acid-binding Ig-like lectin (Siglec) receptors, e.g., Siglec 7, Siglec 9, and/or the like. Additional examples of inhibitory immune receptors which may be inhibited according to the methods of the present disclosure include C-type lectins, including but not limited to: CLEC4A (DCIR), Ly49Q and MICL. Details regarding inhibitory immune receptors may be found, e.g., in Steevels et al. (2011) Eur. J. Immunol. 41(3):575-587. In some embodiments, the cell surface molecule present on the immune cell is a ligand of an inhibitory immune receptor. In certain aspects, the cell surface molecule present on the immune cell is an immune checkpoint molecule. Non-limiting examples of immune checkpoint molecules to which the first moiety may specifically bind include PD-1, PD-L1, CTLA4, TIM3, LAG3, TIGIT, and a member of the B7 family.

According to some embodiments, the bifunctional molecules of the present disclosure include a knottin peptide that specifically binds an extracellular molecule. By “extracellular molecule” is meant a soluble molecule external to the cell membranes of any cells in the vicinity of the soluble molecule. The extracellular molecule may be any extracellular molecule which is desired for targeted degradation via the endosomal/lysosomal pathway. In some embodiments, the extracellular molecule is a ligand for a cell surface receptor. Cell surface receptor ligands of interest include, but are not limited to, growth factors (e.g., epidermal growth factor (EGF), vascular endothelial growth factor (VEGF), and the like), cytokines (e.g., an interleukin, an interferon, a tumor necrosis factor (TNF), a transforming growth factor β (TGF-β), including any particular subtypes of such cytokines), hormones, and the like. In certain aspects, the first moiety specifically binds apolipoprotein E4 (ApoE4).

In some embodiments, the knottin peptide specifically binds an extracellular molecule, where the extracellular molecule is an antibody, e.g., an antibody that specifically binds a cell surface molecule or different extracellular molecule. In some embodiments, the antibody is an autoantibody. Non-limiting examples of autoantibodies include rheumatoid factor (RF), antinuclear antibody (ANA), Antineutrophil Cytoplasmic Antibodies (ANCA), Anti-Double Stranded DNA (anti-dsDNA), Anticentromere Antibodies (ACA), Antihistone Antibodies, Cyclic Citrullinated Peptide Antibodies (CCP), Extractable Nuclear Antigen Antibodies (e.g., anti-SS-A (Ro) and anti-SS-B (La), anti-RNP, anti-Jo-1, anti-Sm, Scl-70), Cardiolipin Antibodies, Beta-2 Glycoprotein 1 Antibodies, Antiphospholipid Antibodies (APA), Lupus anticoagulants (LA), Diabetes-related Autoantibodies, Anti-Tissue Transglutaminase (anti-tTG), Anti-Gliadin Antibodies (AGA), Intrinsic Factor Antibodies, Parietal Cell Antibodies, Thyroid Autoantibodies (e.g., anti-TPO, TSH receptor antibodies), Smooth Muscle Antibodies (SMA), Antimitochondrial Antibodies (AMA), Liver Kidney Microsome Type 1 Antibodies (anti-LKM-1), Anti-Glomerular Basement Membrane (GBM), Acetylcholine Receptor (AChR) Antibodies, etc.

In some embodiments, the knottin peptide specifically binds an extracellular molecule, where the extracellular molecule is a secreted protein that accumulates in disease (e.g., alpha-synuclein), a cholesterol carrier (e.g., ApoB), an infectious disease toxin (e.g., AB toxins, ESAT-6), an infectious particle (e.g., a whole virus, a whole bacterium, etc.), a clotting factor (e.g., Factor IX), the target of any FDA approved antibody that binds to an extracellular molecule (e.g., TNFalpha), any chemokine or cytokine (e.g., mediators of sepsis or chronic inflammation such at IL-1), a proteinaceous hormone (e.g., insulin, ACTH, etc.), a proteinaceous mediator of a mood disorder, a proteinaceous mediator of energy homeostasis (e.g., leptin, ghrelin, etc.), a proteinaceous allergen present in the bloodstream or an antibody against such an allergen (e.g., for peanut allergies), a proteinaceous toxin (e.g., snake venom hyaluronidase, etc.), etc.

In certain embodiments, the knottin peptide specifically binds a cell surface or extracellular molecule, where the cell surface or extracellular molecule is a mutated protein.

In some embodiments, the bifunctional molecule causes shuttling of the mutated protein into the lysosome, promoting its loading onto a major histocompatibility complex (MHC) (e.g., MHC I or MHC II), and thereby promoting recognition of the mutated protein by the immune system. In this context, the bifunctional molecule finds use in generating antibodies specific to a mutated and unwanted protein (e.g., KIT).

By “specifically binds” is meant the first moiety (e.g., knottin peptide or non-knottin integrin-binding moiety) and the second moiety bind to their respective targets with greater affinity, avidity, more readily, and/or with greater duration than they bind to other substances, e.g., in a sample. In some embodiments, the first moiety (e.g., knottin peptide or non-knottin integrin-binding moiety) and the second moiety bind to their respective targets with an affinity or Kₐ (that is, an equilibrium association constant of a particular binding interaction with units of 1/M) of, for example, greater than or equal to about 105 M⁻¹. In certain embodiments, the first moiety and the second moiety bind to their respective targets with a Kₐ greater than or equal to about 10⁶ M⁻¹, 107 M⁻¹, 10⁸ M⁻¹, 109 M⁻¹, 10¹⁰ M⁻¹, 10¹¹ M⁻¹, 10¹² M⁻¹, or 10¹³ M⁻¹. “High affinity” binding refers to binding with a Kₐ of at least 107 M⁻¹, at least 10⁸ M⁻¹, at least 109 M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10-5 M to 10⁻¹³ M, or less). In certain aspects, specific binding means the first moiety and the second moiety bind to their respective targets with a K_{D} of less than or equal to about 10-5 M, less than or equal to about 10⁻⁶ M, less than or equal to about 10-7 M, less than or equal to about 10⁻⁸ M, or less than or equal to about 10-9 M, 10⁻¹⁰ M, 10⁻¹¹ M, or 10⁻¹² M or less. The binding affinity of the first moiety and the second moiety to their respective targets can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

According to some embodiments, when the second moiety specifically binds a lysosomal targeting molecule expressed on the surface of liver cells, e.g., ASGPR, the first moiety specifically binds to a cell surface molecule expressed on hepatocytes (including hepatocellular carcinoma (HCC) cells). Non-limiting examples of such cell surface molecules include cell adhesion receptors (e.g., any of the one or more integrins described elsewhere herein), growth factor receptors, etc. Growth factor receptors of interest include, but are not limited to, epidermal growth factor receptor (EGFR), C-Met, insulin like growth factor 1 receptor (IGF1R), fibroblast growth factor receptor 4 (FGFR4), HER2, and platelet-derived growth factor receptor (PDGFR).

In certain embodiments, the bifunctional molecule finds use in degrading a cell adhesion molecule (e.g., any of the one or more integrins described elsewhere herein), a growth factor receptor, and/or the like, on the surface of hepatocellular carcinoma (HCC) cells (e.g., in vivo upon administration to an individual having HCC to treat the HCC), where the bifunctional molecule comprises a first moiety which is a knottin peptide that specifically binds to one or more integrins, EGFR, C-Met, IGF1R, FGFR4 or HER2, or wherein the first moiety is any suitable moiety that specifically binds to one or more integrins, and wherein the second moiety specifically binds to ASGPR (e.g., the second moiety may comprise a scaffold (e.g., a polymer scaffold) comprising ASGPR ligands, e.g., GaINAc, galactose, and/or glucose).

According to some embodiments, the bifunctional molecule finds use in degrading a cell surface molecule (e.g., growth factor) expressed on fibrotic liver cells (e.g., in vivo upon administration to an individual having fibrosis of the liver), where the bifunctional molecule comprises a first moiety which is a knottin peptide that specifically binds a cell surface or extracellular protein that promotes fibrosis (e.g., PDGFR), and the second moiety specifically binds to ASGPR (e.g., the second moiety may comprise a scaffold (e.g., a polymer scaffold) comprising one or more ASGPR ligands, e.g., GaINAc, galactose, and/or glucose).

In certain embodiments, the bifunctional molecule enhances degradation of the cell surface molecule or extracellular molecule relative to degradation of the cell surface molecule or extracellular molecule in the presence of the first moiety alone. According to some embodiments, the bifunctional molecule enhances degradation of the cell surface molecule or extracellular molecule relative to degradation of the cell surface molecule or extracellular molecule in the presence of the first moiety or the second moiety alone. By “enhances degradation” in this context means the cell surface molecule or extracellular molecule is degraded in the presence of the bifunctional molecule and is not degraded in the presence of the first moiety alone, or the presence of the first moiety or second moiety alone, under the same conditions; or the cell surface molecule or extracellular molecule is degraded in the presence of the bifunctional molecule to a greater extent than the cell surface molecule or extracellular molecule is degraded in the presence of the first moiety alone, or the presence of the first moiety or second moiety alone, under the same conditions. When the cell surface molecule or extracellular molecule is degraded in the presence of the bifunctional molecule to a greater extent than the cell surface molecule or extracellular molecule is degraded in the presence of the first moiety alone, or the presence of the first moiety or second moiety alone under the same conditions, the degradation may be 1.2 fold or greater, 1.4 fold or greater, 1.6 fold or greater, 1.8 fold or greater, 2 fold or greater, 2.5 fold or greater, 3 fold or greater, 3.5 fold or greater, 4 fold or greater, 4.5 fold or greater, 5 fold or greater, 5.5 fold or greater, 6 fold or greater, 6.5 fold or greater, 7 fold or greater, 7.5 fold or greater, 8 fold or greater, 8.5 fold or greater, 9 fold or greater, 9.5 fold or greater, or 10 fold or greater in the presence of the bifunctional molecule.

A bifunctional molecule of the present disclosure may be in any suitable format. In some embodiments, the first moiety is a polypeptide, the second moiety is a polypeptide, and the bifunctional molecule is a fusion protein comprising the first moiety fused to the second moiety. The first moiety may be fused directly to the second moiety. In other aspects, the first moiety may be fused indirectly to the second moiety, e.g., where a spacer domain is disposed between the first and second moieties. Also provided by the present disclosure are nucleic acids that encode the bifunctional molecule when the bifunctional molecule is a fusion protein. Expression vectors that include such nucleic acids are also provided, as are cells (e.g., host cells) that include any of the nucleic acids and/or expression vectors of the present disclosure. Also provided are methods of producing such cells, the methods including introducing into a cell any of the nucleic acids and/or expression vectors of the present disclosure, e.g., using a suitable cell transfection protocol and transfection reagents. Also provided by the present disclosure are methods of making the bifunctional molecule when the bifunctional molecule is a fusion protein. Such methods may include culturing a cell of the present disclosure under conditions in which the bifunctional molecule is expressed in the cell.

Other suitable formats for the bifunctional molecules of the present disclosure include conjugates. Accordingly, in some embodiments, a bifunctional molecule of the present disclosure includes the first moiety conjugated to the second moiety. In certain embodiments, the first moiety is knottin peptide or integrin-binding first moiety (which may be a knottin peptide) and the second moiety specifically binds M6PR. By way of example, the first moiety may be a knottin peptide or integrin-binding first moiety (which may be a knottin peptide) and the second moiety may include a polymer scaffold that includes/displays one or more M6PR ligands, e.g., one or more M6Ps and/or M6P analogs, where the polymer scaffold is conjugated to the first moiety. In some such embodiments, the second moiety is a glycoprotein including one or more amino acids functionalized with the one or more M6PR ligands. Methods of making such conjugates are also provided, the methods including conjugating the first moiety to the second moiety. In some embodiments, the methods include site-specifically conjugating the first moiety to the second moiety. For example, when the first moiety includes a peptide, the conjugating may include site-specifically conjugating the second moiety to a pre-selected amino acid of the first moiety. In certain aspects, the pre-selected amino acid is at the N-terminus or C-terminus of the first moiety. In other aspects, the pre-selected amino acid is internal to the first moiety—that is, between the N-terminal and C-terminal amino acid of the first moiety. In some embodiments, the pre-selected amino acid is a non-natural amino acid. Non-limiting examples of non-natural amino acids which may be provided to the first and/or second moieties to facilitate conjugation include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated and selected to provide a functional group of interest are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere.

In some embodiments, conjugating the first moiety to the second moiety includes conjugating the second moiety to a glycan on the first moiety, or vice versa. Such a method may include modifying one or more glycans on the first moiety to provide a functional group to which the second moiety may be attached. In one non-limiting example, N-glycans on the first moiety may be modified via periodate oxidation to aldehyde groups, which could then be functionalized with the second moiety, e.g., aminooxy M6Pn.

When the bifunctional molecule is a conjugate, one or more linkers may be employed to facilitate conjugation of the first moiety to the second moiety. Non-limiting examples of such linkers include ester linkers, amide linkers, maleimide or maleimide-based linkers; valine-citrulline linkers; hydrazone linkers; N-succinimidyl-4-(2-pyridyldithio)butyrate (SPDB) linkers; Succinimidyl-4-(N-maleimidomethyl)cyclohexane-1-carboxylate (SMCC) linkers; vinylsulfone-based linkers; linkers that include polyethylene glycol (PEG), such as, but not limited to tetraethylene glycol; linkers that include propanoic acid; linkers that include caproleic acid, and linkers including any combination thereof. In certain aspects, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. According to certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13.

Numerous strategies are available for conjugating the first and second moieties through a linker. For example, the first moiety may be derivatized by covalently attaching the linker to the first moiety, where the linker has a functional group capable of reacting with a “chemical handle” on the second moiety. Also by way of example, the second moiety may be derivatized by covalently attaching the linker to the second moiety, where the linker has a functional group capable of reacting with a “chemical handle” on the first moiety. The functional group on the linker may vary and may be selected based on compatibility with the chemical handle on the first or second moiety. According to one embodiment, the chemical handle is provided by incorporation of an unnatural amino acid having the chemical handle into the first or second moiety. In some embodiments, conjugating the first and second moieties is by alkyne-azide cycloaddition.

Other suitable formats for the bifunctional molecules of the present disclosure include bispecific antibodies. For example, a bifunctional molecule of the present disclosure may be a bispecific antibody where the first moiety (e.g., a first Fab arm) specifically binds one or more integrins, and the second moiety (e.g., a second Fab arm) specifically binds a lysosomal targeting molecule (e.g., M6PR). In some embodiments, the bispecific antibody disengages from the target at the lowered pH of the endosomes. Such a strategy allows for a given bispecific antibody to cycle with the receptor and continuously deliver cargo and targets to the lysosome, without degradation of the antibody. Approaches for making bispecific antibodies are known. For example, when the bifunctional molecule is a bispecific antibody, the bispecific antibody may be made using a “knobs-into-holes” (KIHs) approach. KIHs technology involves engineering CH3 domains to create either a “knob” or a “hole” in each heavy chain to promote heterodimerization. KIHs design and production strategies are known and include those described, e.g., in Xu et al. (2015) MAbs 7(1):231-42; Carter et al. (2001) J. Immunol. Methods 248(1-2):7-15; Ridgway et al. (1996) Protein Eng. 9(7):617-2; and Merchant et al. (1998) Nat. Biotechnol. 16(7):677-81.

### Compositions

As summarized above, the present disclosure provides compositions. The compositions may include any of the bifunctional molecules of the present disclosure, including any of the bifunctional molecules described in the Bifunctional Molecules section above or in the Experimental section below, which sections are incorporated but not reiterated herein for purposes of brevity.

In certain aspects, the compositions include a bifunctional molecule of the present disclosure present in a liquid medium. The liquid medium may be an aqueous liquid medium, such as water, a buffered solution, and the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a protease inhibitor, glycerol, and the like may be present in such compositions.

Pharmaceutical compositions are also provided. The pharmaceutical compositions include any of the bifunctional molecules of the present disclosure, and a pharmaceutically-acceptable carrier. The pharmaceutical compositions generally include a therapeutically effective amount of the bifunctional molecule. By “therapeutically effective amount” is meant a dosage sufficient to produce a desired result, e.g., an amount sufficient to effect beneficial or desired therapeutic (including preventative) results, such as a reduction in cellular proliferation in an individual having a cell proliferative disorder (e.g., cancer) associated with the cell surface molecule (e.g., integrin) or extracellular molecule to which the first moiety of the bifunctional molecule specifically binds, etc. An effective amount may be administered in one or more administrations.

A bifunctional molecule of the present disclosure can be incorporated into a variety of formulations for therapeutic administration. More particularly, the bifunctional molecule can be formulated into pharmaceutical compositions by combination with appropriate pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the bifunctional molecules of the present disclosure suitable for administration to an individual (e.g., suitable for human administration) are generally sterile and may further be free of detectable pyrogens or other contaminants contraindicated for administration to an individual according to a selected route of administration.

In pharmaceutical dosage forms, the bifunctional molecule can be administered alone or in appropriate association, as well as in combination, with other pharmaceutically-active compounds. The following methods and excipients are merely examples and are in no way limiting.

For oral preparations, the bifunctional molecule can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The bifunctional molecules can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The pharmaceutical composition may be in a liquid form, a lyophilized form or a liquid form reconstituted from a lyophilized form, where the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the bifunctional molecule may be prepared in a pH-buffered solution, e.g., at pH ranging from about 4.0 to about 8.0, such as from about 4.5 to about 7.5, e.g., from about 5.0 to about 7.0. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

### Methods of Use

As summarized above, also provides are methods of using the bifunctional molecules of the present disclosure. In some embodiments, the methods including using any of the bifunctional molecules described in the Bifunctional Molecule section above or the Experimental section below, which sections are incorporated but not reiterated herein for purposes of brevity.

In certain aspects, provided are methods of degrading a cell surface molecule or extracellular molecule. Such methods include contacting the cell surface molecule (e.g., one or more integrins) or extracellular molecule with any of the bifunctional molecules of the present disclosure, under conditions in which the lysosomal targeting molecule shuttles the cell surface molecule or extracellular molecule to the lysosome for degradation. Such methods find use in a variety of applications. In certain aspects, the method is performed in vitro (e.g., in a tube, cell culture plate or well, or the like) and finds use, e.g., in testing and/or research applications. In other aspects, the method is performed in vivo (e.g., in an individual to whom the bifunctional molecule is administered) and finds use, e.g., in clinical/therapeutic applications, including but not limited to, methods of treating a cell proliferative disorder (e.g., cancer) in an individual in need thereof.

In some embodiments, provided are methods that include administering to an individual in need thereof a therapeutically effective amount of any of the bifunctional molecules or any of the pharmaceutical compositions of the present disclosure. A variety of individuals are treatable according to the subject methods. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human.

In some embodiments, an effective amount of the bifunctional molecule (or pharmaceutical composition including same) is an amount that, when administered alone (e.g., in monotherapy) or in combination (e.g., in combination therapy) with one or more additional therapeutic agents, in one or more doses, is effective to reduce the symptoms of a medical condition of the individual (e.g., a cell proliferative disorder (e.g., cancer), a neurodegenerative disorder, etc.) by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the bifunctional molecule or pharmaceutical composition.

In certain aspects, the individual has, or is suspected of having, a neurodegenerative disorder characterized by amyloid-β deposition in the brain (e.g., Alzheimer's Disease) or tau protein deposition in the brain (e.g., a tauopathy), and the first moiety of the bifunctional molecule is a knottin peptide that specifically binds to apoE4 (e.g., apoE4 expressed from the E4 allele of the APOE4 gene), such that targeted degradation of apoE4 treats the individual's neurodegenerative disorder.

In some embodiments, provided are methods that include administering to an individual having a cell proliferative disorder (e.g., cancer) a therapeutically effective amount of any of the bifunctional molecules or any of the pharmaceutical compositions of the present disclosure. According to such methods, the first moiety of the bifunctional molecule specifically binds a cell surface molecule or extracellular molecule that at least contributes to individual's cancer, and where targeted degradation of the cell surface molecule or extracellular molecule using the bifunctional molecule treats the individual's cancer. In certain aspects, the first moiety specifically binds to a molecule selected from a cell surface molecule on a cancer cell, a ligand for a cell surface molecule on a cancer cell, a cell surface molecule on an immune cell, a ligand for a cell surface molecule on an immune cell, an inhibitory immune receptor, and a ligand of an inhibitory immune receptor.

In certain embodiments, the individual has a cancer characterized by the presence of a solid tumor, a semi-solid tumor, a primary tumor, a metastatic tumor, or the like. In some embodiments, the individual has a cancer selected from breast cancer, melanoma, lung cancer, colorectal cancer, prostate cancer, glioma, bladder cancer, endometrial cancer, kidney cancer, leukemia (e.g., acute myeloid leukemia (AML)) liver cancer (e.g., hepatocellular carcinoma (HCC), such as primary or recurrent HCC), non-Hodgkin lymphoma, pancreatic cancer, thyroid cancer, any combinations thereof, and any sub-types thereof.

According to some embodiments, the individual has a particular liver disease (including but not limited to hepatocellular carcinoma (HCC)), and the methods are for treating the disease. For example, in certain embodiments, the individual has HCC, the first moiety binds a cell surface molecule on HCC cells of the individual, and the second moiety binds ASGPR. In certain embodiments, the first moiety binds a tumor-promoting protein on HCC cells of the individual. According to some embodiments, the tumor-promoting protein is one or more integrins, or a growth factor, on the HCC cells. Non-limiting examples of such integrins include αvβ1 integrin, αvβ3 integrin, αvβ5 integrin, αvβ6 integrin, α5β1 integrin, and any combination thereof. Non-limiting examples of such growth factors include EGFR, C-Met, IGF1R, and FGFR4. Such a bifunctional molecule may include any of the second moieties that bind ASGPR as described elsewhere herein.

In certain embodiments, the individual has fibrosis of the liver, and the methods are for treating the liver fibrosis. For example, according to some embodiments, the individual has liver fibrosis, the first moiety binds a cell surface molecule on fibrotic liver cells of the individual, and the second moiety binds ASGPR. In certain embodiments, the first moiety binds a fibrosis-promoting protein on the fibrotic liver cells of the individual. According to some embodiments, the fibrosis-promoting protein is a growth factor on the fibrotic liver cells. A non-limiting example of such a growth factor is PDGFR. Such a bifunctional molecule may include any of the second moieties that bind ASGPR as described elsewhere herein.

In any of the methods of using the bifunctional molecules of the present disclosure, in certain embodiments, the bifunctional molecule enhances degradation of the cell surface molecule or extracellular molecule relative to degradation of the cell surface molecule or extracellular molecule in the presence of the first moiety alone. Similarly, in any of the methods of using the bifunctional molecules of the present disclosure, according to some embodiments, the bifunctional molecule enhances degradation of the cell surface molecule or extracellular molecule relative to degradation of the cell surface molecule or extracellular molecule in the presence of the first moiety or the second moiety alone. Details regarding such enhancement of degradation are provided in the Bifunctional Molecules section above and incorporated but not reiterated herein for purposes of brevity.

By “treat”, “treating” or “treatment” is meant at least an amelioration of the symptoms associated with the medical condition (e.g., cell proliferative disorder, e.g., cancer) of the individual, where amelioration is used in a broad sense to refer to at least a reduction in the magnitude of a parameter, e.g., symptom, associated with the medical condition being treated. As such, treatment also includes situations where the medical condition, or at least symptoms associated therewith, are completely inhibited, e.g., prevented from happening, or stopped, e.g., terminated, such that the individual no longer suffers from the medical condition, or at least the symptoms that characterize the medical condition.

In certain aspects, the present disclosure provides methods of enhancing antibody-dependent cellular cytotoxicity (ADCC) including administering to an individual in need of ADCC a bifunctional molecule or pharmaceutical composition of the present disclosure. In some embodiments, the first moiety of the bifunctional molecule is a knottin peptide that specifically binds to an inhibitory immune receptor or a ligand of an inhibitory immune receptor. In certain aspects, the first moiety of the bifunctional molecule is a knottin peptide that specifically binds to an immune checkpoint molecule such as PD-1, PD-L1, CTLA4, TIM3, LAG3, TIGIT, or a member of the B7 family.

The bifunctional molecule or pharmaceutical composition may be administered to the individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, nasal, oral, and other enteral and parenteral routes of administration. In some embodiments, the administering is by parenteral administration. Routes of administration may be combined, if desired, or adjusted depending upon the bifunctional molecule and/or the desired effect. The bifunctional molecules or pharmaceutical compositions may be administered in a single dose or in multiple doses. In some embodiments, the bifunctional molecule or pharmaceutical composition is administered intravenously. In some embodiments, the bifunctional molecule or pharmaceutical composition is administered by injection, e.g., for systemic delivery (e.g., intravenous infusion) or to a local site, e.g., intratumorally.

### Kits

As summarized above, the present disclosure also provides kits. In some embodiments, a subject kit includes any of the bifunctional molecules of the present disclosure (including any of the bifunctional molecules described in the Bifunctional Molecule section above, which is incorporated but not reiterated herein for purposes of brevity), and instructions for using the bifunctional molecule to degrade the cell surface molecule or extracellular molecule to which the first moiety specifically binds. In certain aspects, the instructions are for degrading the cell surface molecule or extracellular molecule in vitro, e.g., for research and/or testing purposes. In other aspects, the instructions are for degrading the cell surface molecule or extracellular molecule in vivo, e.g., for clinical/therapeutic applications. For example, provided are kits that include any of the bifunctional molecules or pharmaceutical compositions of the present disclosure, and instructions for administering the bifunctional molecule or pharmaceutical composition to an individual in need thereof. Such kits may include a quantity of the bifunctional molecule or pharmaceutical composition, present in unit dosages, e.g., ampoules, or a multi-dosage format. As such, in certain embodiments, the kits may include one or more (e.g., two or more) unit dosages (e.g., ampoules) of the bifunctional molecule or pharmaceutical composition.

The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular bifunctional molecule employed, the effect to be achieved, and the pharmacodynamics associated with the bifunctional molecule, in the individual. In yet other embodiments, the kits may include a single multi dosage amount of the bifunctional molecule or pharmaceutical composition.

In other aspects, provided are kits that include any of the glycopolymers of the present disclosure (including any of the glycopolymers described in the Bifunctional Molecule section above, which is incorporated but not reiterated herein for purposes of brevity), and instructions for conjugating the glycopolymer to a molecule of interest. Such kits may further include reagents for conjugating the glycopolymer to a molecule of interest. In some embodiments, the molecule of interest is a polypeptide. Non-limiting examples of such polypeptides include antibodies. In certain aspects, the molecule of interest specifically binds a cell surface molecule or extracellular molecule, including any of the cell surface molecules or extracellular molecules described in the Bifunctional Molecule section above, which is incorporated but not reiterated herein for purposes of brevity.

Components of the kits may be present in separate containers, or multiple components may be present in a single container.

The instructions included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Glycopolymers, Monomers, and Methods of Making Same

The present disclosure also provides glycopolymers. In some embodiments, a glycopolymer of the present disclosure includes a polymer scaffold and one or more mannose-6-phosphate receptor (M6PR) ligands attached to the polymer scaffold. The glycopolymer, polymer scaffold, and/or M6PR ligands may be any of those described in the Bifunctional Molecule section above, which is incorporated but not reiterated herein for purposes of brevity. For example, the glycopolymer may be a glycoprotein including one or more amino acids functionalized with the one or more M6PR ligands. The glycoprotein may be a N-carboxyanhydride (NCA)-derived glycoprotein. In certain aspects, the one or more M6PR ligands include one or more mannose-6-phosphates (M6P). Alternatively, or additionally, the one or more M6PR ligands include one or more M6P analogs, e.g., any of the M6P analogs described herein, such as one or more mannose-6-phosphonates (M6Pn). In some embodiments, the polymer scaffold includes from 1 to 50 M6PR ligands, such as from 1 to 40, 1 to 30, 1 to 20, 1 to 10 (e.g., 1 to 6), or 1 to 5 M6PR ligands. In certain aspects, the polymer scaffold includes from 10 to 50, 15 to 45, 20 to 40, or 25 to 35 M6PR ligands. In certain aspects, the polymer scaffold includes 5 or more, 10 or more, 20 or more, 30 or more, or 40 or more M6PR ligands.

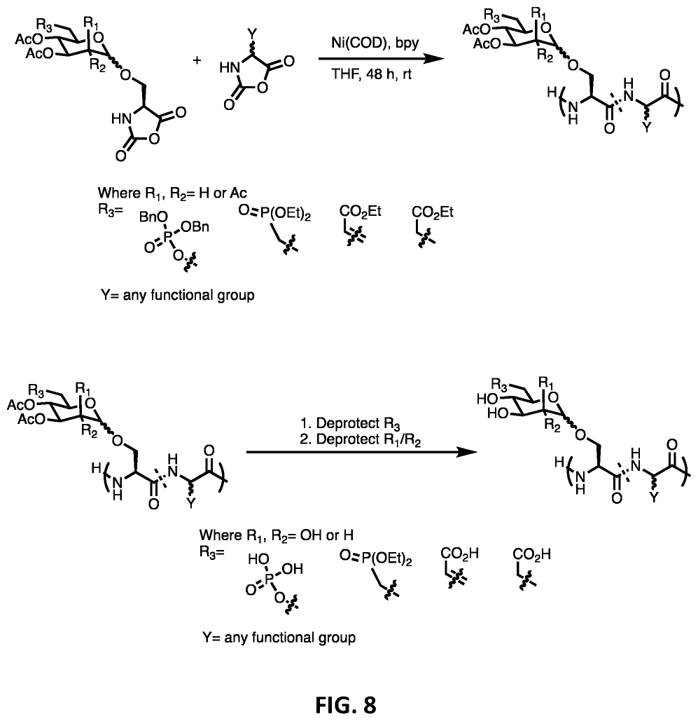

Also provided are methods of making the glycopolymers of the present disclosure. In some embodiments, making the glycopolymer includes polymerization. The polymerization may be by NCA polymerization, an example of which is schematically illustrated in FIG. 8.

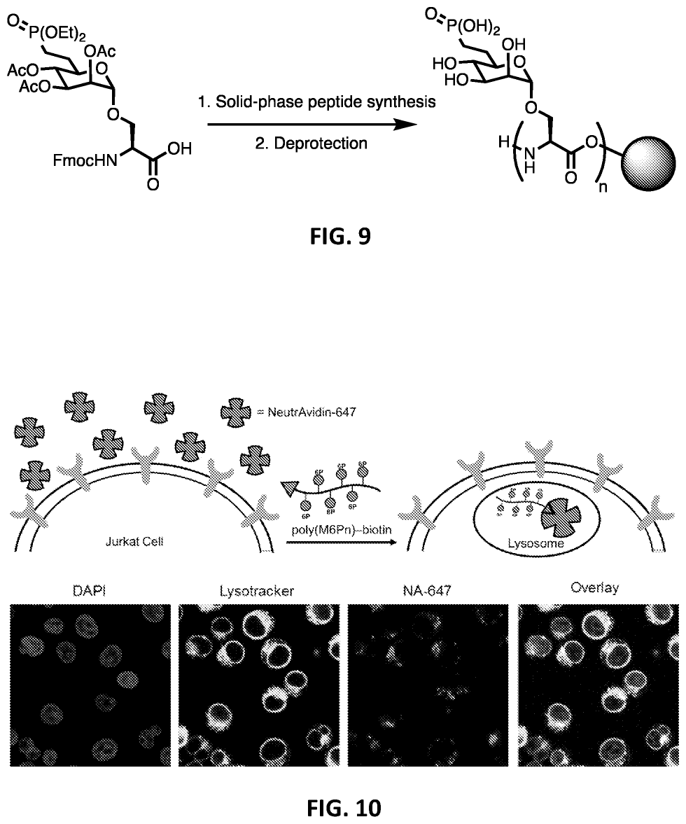

In certain aspects, a method of making the glycopolymer includes attaching the one or more M6PR ligands to the polymer scaffold. In other aspects, such methods include synthesizing the polymer scaffold from monomers functionalized with the one or more M6PR ligands. For example, the scaffold may be synthesized from one or more monomers functionalized with the one or more M6PR ligands, where the synthesizing is by solid-phase synthesis. An example solid-phase synthesis scheme is provided in FIG. 9.

In related aspects, the present disclosure provides monomers. The monomers are functionalized with one or more mannose-6-phosphate receptor (M6PR) ligands. In certain aspects, the monomers are amino acids. In some embodiments, the monomers are non-natural amino acids. The one or more M6PR ligands may include one or more mannose-6-phosphates (M6P). Alternatively, or additionally, the one or more M6PR ligands may include one or more M6P analogs, e.g., any of the M6P analogs described herein, such as mannose-6-phosphonates (M6Pn).

Notwithstanding the appended claims, the present disclosure is also defined by the following embodiments.

1. A bifunctional molecule comprising:
a first moiety that specifically binds a cell surface molecule, wherein the first moiety is a knottin peptide comprising an engineered loop that binds to the cell surface molecule; and
a second moiety that specifically binds a lysosomal targeting molecule.
   2. The bifunctional molecule of embodiment 1, wherein the bifunctional molecule reduces cell surface abundance of the cell surface molecule relative to the cell surface abundance of the cell surface molecule in the presence of the first moiety alone.
   3. The bifunctional molecule of embodiment 1 or embodiment 2, wherein the bifunctional molecule enhances degradation of the cell surface molecule relative to degradation of the cell surface molecule in the presence of the first moiety alone.
   4. The bifunctional molecule of any one of embodiments 1 to 3, wherein the knottin peptide is selected from the group consisting of: an EETI-II peptide, an AgRP peptide, a ω-conotoxin peptide, a Kalata B1 peptide, an MCoTI-II peptide, an agatoxin peptide, and a chlorotoxin peptide.
   5. The bifunctional molecule of any one of embodiments 1 to 4, wherein the cell surface molecule is a cell surface receptor.
   6. The bifunctional molecule of embodiment 5, wherein the cell surface receptor is a cell adhesion receptor.
   7. The bifunctional molecule of embodiment 6, wherein the cell adhesion receptor is an integrin.
   8. The bifunctional molecule of embodiment 7, wherein the integrin is selected from the group consisting of: αvβ1 integrin, αvβ3 integrin, αvβ5 integrin, αvβ6 integrin, α5β1 integrin, and any combination thereof.
   9. The bifunctional molecule of embodiment 7 or embodiment 8, wherein the knottin peptide is an integrin-binding knottin peptide comprising an amino acid having 70% or greater, 75% or greater, 80% or greater, 85% or greater, 90% or greater, 95% or greater, or 100% identity to the amino acid sequence

(2.5D)
(SEQ ID NO: 7)
GCPQGRGDWAPTSCKQDSDCRAGCVCGPNGFCG
or
(2.5F)
(SEQ ID NO: 8)
GCPRPRGDNPPLTCKQDSDCLAGCVCGPNGXCG.

10. The bifunctional molecule of any one of embodiments 1 to 9, wherein the cell surface molecule is present on a cancer cell.
11. A bifunctional molecule comprising:
a first moiety that specifically binds an extracellular molecule, wherein the first moiety is a knottin peptide comprising an engineered loop that binds to the extracellular molecule; and
a second moiety that specifically binds a lysosomal targeting molecule.
   12. The bifunctional molecule of embodiment 11, wherein the extracellular molecule is a ligand for a cell surface receptor.
   13. The bifunctional molecule of embodiment 12, wherein the extracellular molecule is a growth factor.
   14. The bifunctional molecule of embodiment 12, wherein the extracellular molecule is a cytokine or a chemokine.
   15. The bifunctional molecule of any one of embodiments 1 to 14, wherein the knottin peptide is fused to a heterologous protein.
   16. The bifunctional molecule of embodiment 15, wherein the knottin peptide is fused at its N- or C-terminus to heterologous protein.
   17. The bifunctional molecule of embodiment 15 or embodiment 16, wherein the heterologous protein is an Fc region.
   18. The bifunctional molecule of embodiment 15 or embodiment 16, wherein the heterologous protein binds to a cell surface molecule different from the cell surface molecule bound by the knottin peptide.
   19. The bifunctional molecule of any one of embodiments 15 to 17, wherein the knottin polypeptide is fused to a second heterologous protein.
   20. A bifunctional molecule comprising:
a first moiety that specifically binds an integrin; and
a second moiety that specifically binds a lysosomal targeting molecule.
   21. The bifunctional molecule of embodiment 20, wherein the integrin is selected from the group consisting of: αvβ1 integrin, αvβ3 integrin, αvβ5 integrin, αvβ6 integrin, α5β1 integrin, and any combination thereof.
   22. The bifunctional molecule of any one of embodiments 1 to 21, wherein the second moiety is selected from the group consisting of: a polypeptide, a ligand, an aptamer, a nanoparticle, and a small molecule.
   23. The bifunctional molecule of any one of embodiments 1 to 22, wherein the lysosomal targeting molecule is a mannose-6-phosphate receptor (M6PR).
   24. The bifunctional molecule of embodiment 23, wherein the second moiety comprises one or more M6PR ligands.
   25. The bifunctional molecule of embodiment 24, wherein the one or more M6PR ligands comprise one or more mannose-6-phosphates (M6P).
   26. The bifunctional molecule of embodiment 24 or embodiment 25, wherein the one or more M6PR ligands comprise one or more M6P analogs.
   27. The bifunctional molecule of embodiment 26, wherein the one or more M6P analogs comprise one or more mannose-6-phosphonates (M6Pn).
   28. The bifunctional molecule of any one of embodiments 24 to 27, wherein the second moiety comprises from 1 to 500 M6PR ligands.
   29. The bifunctional molecule of any one of embodiments 24 to 28, wherein the second moiety comprises a polymer scaffold that displays the one or more M6PR ligands.
   30. The bifunctional molecule of embodiment 29, wherein the polymer scaffold is a glycopolymer comprising the one or more M6PR ligands.
   31. The bifunctional molecule of embodiment 30, wherein the glycopolymer is a glycoprotein comprising one or more amino acids functionalized with the one or more M6PR ligands.
   32. The bifunctional molecule of embodiment 31, wherein the glycoprotein is a N-carboxyanhydride (NCA)-derived glycoprotein.
   33. The bifunctional molecule of any one of embodiments 1 to 22, wherein the lysosomal targeting molecule is expressed on the surface of liver cells.
   34. The bifunctional molecule of embodiment 33, wherein the lysosomal targeting molecule is expressed on the surface of hepatocytes.
   35. The bifunctional molecule of embodiment 33 or embodiment 34, wherein the lysosomal targeting molecule is expressed on the surface of hepatocellular carcinoma (HCC) cells, fibrotic liver cells, or both.
   36. The bifunctional molecule of any one of embodiments 33 to 35, wherein the lysosomal targeting molecule is asialoglycoprotein receptor (ASGPR).
   37. The bifunctional molecule of embodiment 36, wherein the second moiety comprises one or more ASGPR ligands.
   38. The bifunctional molecule of embodiment 37, wherein the one or more ASGPR ligands comprises one or more N-acetylgalactosamines (GaINAc).
   39. The bifunctional molecule of embodiment 37 or embodiment 38, wherein the one or more ASGPR ligands comprises one or more galactoses.
   40. The bifunctional molecule of any one of embodiments 37 to 39, wherein the one or more ASGPR ligands comprises one or more glucoses.
   41. The bifunctional molecule of any one of embodiments 37 to 40, wherein the second moiety comprises from 1 to 500 ASGPR ligands.
   42. The bifunctional molecule of any one of embodiments 37 to 41, wherein the second moiety comprises a polymer comprising the one or more ASGPR ligands.
   43. The bifunctional molecule of embodiment 42, wherein the second moiety comprises poly(GaINAc-co-Ala).
   44. The bifunctional molecule of embodiment 38, wherein the second moiety comprises a monovalent, bivalent, or trivalent GaINAc-containing dendrimer scaffold.
   45. The bifunctional molecule of embodiment 44, wherein the second moiety comprises a trivalent GaINAc-containing dendrimer scaffold.
   46. The bifunctional molecule of embodiment 39, wherein the second moiety comprises a monovalent, bivalent, or trivalent galactose-containing dendrimer scaffold.
   47. The bifunctional molecule of embodiment 46, wherein the second moiety comprises a trivalent galactose-containing dendrimer scaffold.
   48. The bifunctional molecule of any one of embodiments 1 to 47, wherein the bifunctional molecule is a conjugate comprising the first moiety conjugated to the second moiety.
   49. A method of degrading a cell surface molecule, comprising:
contacting the cell surface molecule with the bifunctional molecule of any one of embodiments 1 to 48 under conditions in which the lysosomal targeting molecule shuttles the cell surface molecule to the lysosome for degradation.
   50. The method according to embodiment 49, wherein the bifunctional molecule enhances degradation of the cell surface molecule relative to degradation of the cell surface molecule in the presence of the first moiety alone.
   51. The method according to embodiment 49 or embodiment 50, wherein the method is performed in vitro.
   52. The method according to embodiment 49 or embodiment 50, wherein the method is performed in vivo.
   53. The method according to any one of embodiments 49 to 52, wherein the cell surface molecule is an integrin.
   54. The method according to embodiment 53, wherein the integrin is selected from the group consisting of: avP1 integrin, avP3 integrin, avP5 integrin, avP6 integrin, α5β1 integrin, and any combination thereof.
   55. A pharmaceutical composition comprising:
the bifunctional molecule of any one of embodiments 1 to 48; and
a pharmaceutically acceptable carrier.
   56. The pharmaceutical composition of embodiment 55, wherein the composition is formulated for parenteral administration.
   57. A method comprising administering to an individual in need thereof the pharmaceutical composition of embodiment 55 or embodiment 56.
   58. A method of treating cancer comprising administering to an individual having cancer an effective amount of the pharmaceutical composition of embodiment 55 or embodiment 56.
   59. A kit comprising:
the bifunctional molecule of any one of embodiments 1 to 48; and
instructions for degrading the cell surface molecule to which the first moiety specifically binds.
   60. The kit of embodiment 59, wherein the instructions are for degrading the cell surface molecule or extracellular molecule in vitro.
   61. The kit of embodiment 59, wherein the instructions are for degrading the cell surface molecule or extracellular molecule in vivo.
   62. A kit comprising:
the bifunctional molecule of any one of embodiments 1 to 48 or the pharmaceutical composition of embodiment 55 or embodiment 56; and
instructions for administering the bifunctional molecule or pharmaceutical composition to an individual in need thereof.
   63. The kit of embodiment 62, wherein the bifunctional molecule or pharmaceutical composition is present in one or more unit dosages.
   64. The kit of embodiment 62, wherein the bifunctional molecule or pharmaceutical composition is present in two or more unit dosages.

The following examples are offered by way of illustration and not by way of limitation.

### EXPERIMENTAL

### Example 1—Mannose-6-Phosphate Polymers Shuttle Cargo to Lysosomes

Tested in this example was a bifunctional molecule (see FIG. 10) that includes a biotin cap (the “first moiety” as used herein—depicted as a triangle in FIG. 10) and an M6Pn polymer (the “second moiety” as used herein) to determine whether the bifunctional molecule could mediate transfer of NeutrAvidin-647 (NA647—a protein to which biotin strongly binds) to lysosomes from the extracellular space for degradation. FIG. 10 provides fluorescence imaging results (bottom) demonstrating that the bifunctional molecule can indeed mediate transfer of NeutrAvidin-647 to lysosomes, as colocalization of both protein and lysosome staining dye are observed.

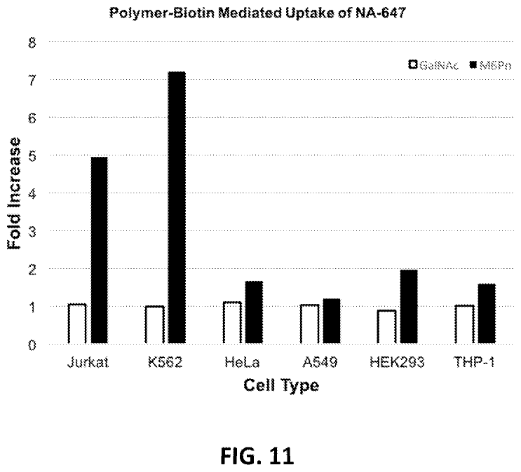

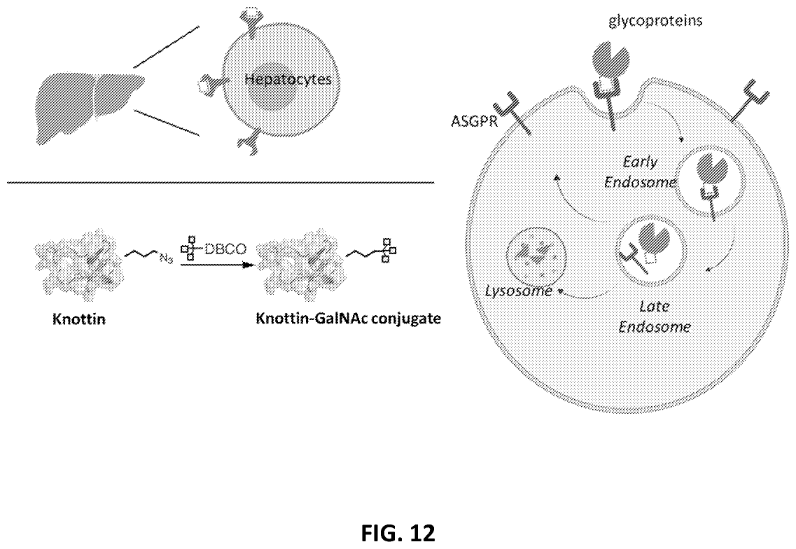

Next, various cell lines were tested in a manner as described above. Shown in FIG. 11 is data demonstrating that several cell lines exhibit uptake of NA647 in a M6Pn polymer-dependent manner. In view of these results, it is expected that any cell line bearing M6PRs (e.g., CIM6PRs) will allow for shuttling of cell surface and extracellular molecules to the lysosome by this method, and is not limited to the cell lines tested in the present study.

### Example 2—Knottin-Based LYTACs Potentiate Antiproliferation

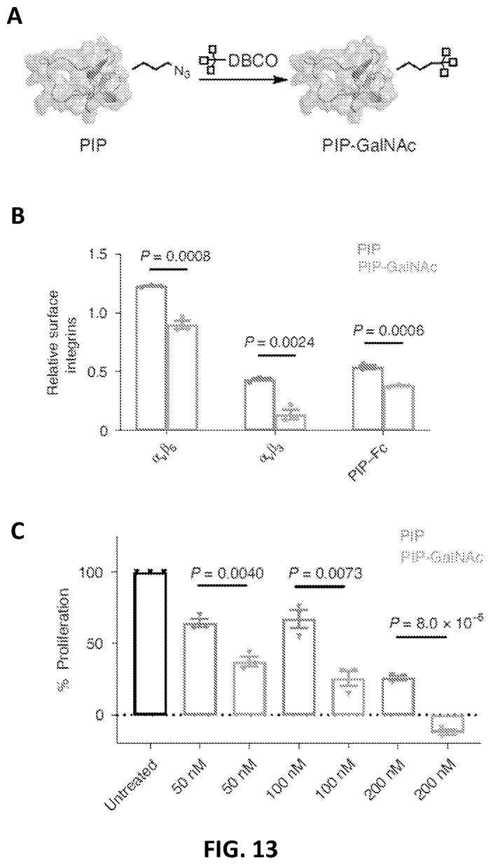

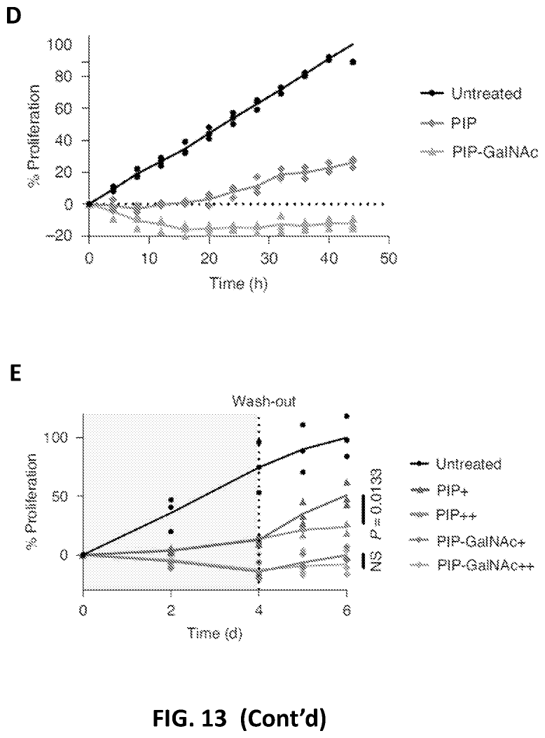

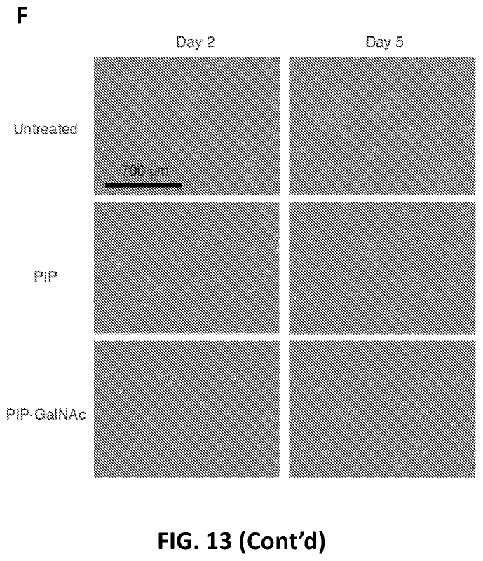

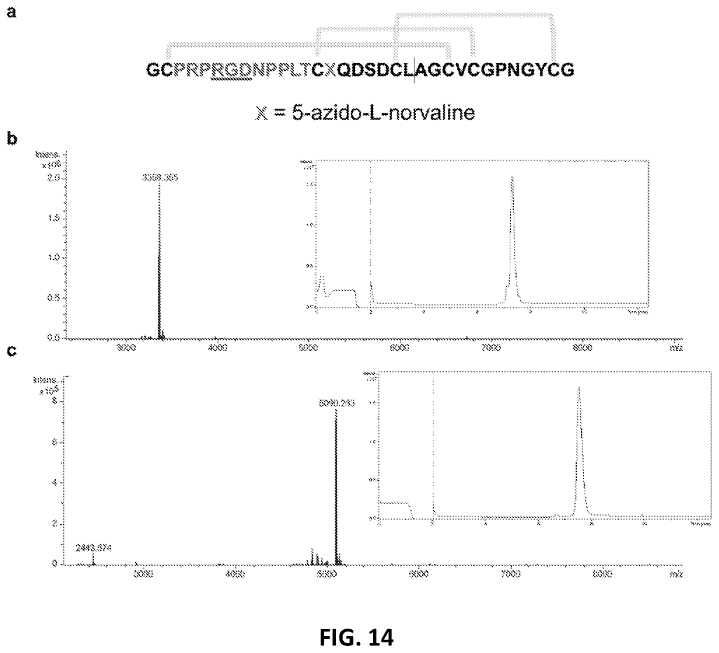

Previous LYTACs targeting membrane proteins were based on antibody or antibody fragments as the target binders. Although antigen-binding fragments (Fab) non-specifically conjugated to several M6Pn polymers could induce degradation, investigated in the present study was whether a knottin peptide binder conjugated to a single ASGPR ligand can promote degradation and induce enhanced functional consequences. Used to test this was a targeting ligand composed of a polyspecific integrin-binding knottin peptide (PIP), also known as EETI 2.5F, that was previously engineered to bind several tumor-associated integrins with high affinity. In particular, PIP binds to αvβ31, αvβ3, αvβ5, αvβ6 and α5β1 integrins, which are known to be overexpressed in various cancer types and facilitate proliferation, migration and metastasis. Given that the 3.4-kDa PIP peptide is produced by solid-phase peptide synthesis, azido-I-norvaline was readily incorporated into the sequence to enable site-specific conjugation via a single tri-GaINAc-DBCO moiety, resulting in PIP—GaINAc (FIG. 13A and FIG. 14).

The surface levels of integrins were measured by flow cytometry following treatment in HEPG2 cells. It was observed that PIP—GaINAc depleted cell surface αvβ3 integrin levels threefold relative to PIP treatment alone. Treatment with PIP increased the expression of integrin αvβ5 while PIP—GaINAc overturned this effect, resulting in modest degradation. Measurement of additional integrins that interact with PIP was challenging due to their low surface expression or a lack of a specific detection antibody. Nonetheless, the total surface level of integrins that bind to PIP was determined by utilizing a PIP-Fc fusion for staining of the Fc fragment. Kwan et al. (2017) J. Exp. Med. 214:1679-1690. Detection of PIP-Fc revealed that PIP—GaINAc depleted about 60% of integrins while PIP alone degraded 40% (FIG. 13B).

Assessed next was whether PIP—GaINAc has an effect on the proliferation of HEPG2 cells compared to the parent peptide. PIP—GaINAc was substantially more effective at inhibiting proliferation than PIP at various concentrations and durations of treatment (FIG. 13C and FIG. 13D). It was validated that tri-GaINAc must be conjugated to PIP to potentiate antiproliferation, as co-incubation of PIP with an equimolar concentration of exogenous tri-GaINAc exhibited similar effects as incubation with PIP alone. To verify the involvement of ASGPR on enhanced antiproliferation, cells were co-incubated with asialofetuin (ASF), which is a commonly used inhibitor of ASGPR. ASF ablated the enhanced antiproliferative effect of PIP—GaINAc, confirming that PIP—GaINAc must engage ASGPR. The antiproliferative phenotype induced by PIP—GaINAc persisted after conjugate wash-out, while wash-out rescued proliferation in PIP-treated cells. The unexpected long-acting inhibition of cell growth by PIP—GaINAc indicates that continuous incubation is not required for modulating the phenotypic consequences resulting from cell surface depletion of targets (FIG. 13E). Moreover, cells treated with PIP—GaINAc displayed noticeable morphological changes. Untreated and PIP-treated cells generally displayed epithelial-like clusters, whereas the PIP—GaINAc-treated cells formed smaller, circular clusters over the course of 5 days following treatment (FIG. 13F). Previous work demonstrated that αvβ3 integrin expression is associated with tumor size, invasion and metastasis of HCC, and that antisense gene transfections of αv and β3 integrin reduced proliferation in HEPG2 cells. The present results strongly suggest that increased degradation of integrins by LYTACs can result in remarkably enhanced functional effects compared to the parent binder alone.

### Materials and Methods for Example 2

Generation of PIP—GaNAc—Synthesis of PIP

Solid-phase peptide synthesis, peptide cleavage and folding methods are described in previous work that utilized azide-modified knottin synthesis. See Cox et al. (2016) Angew. Chem. Int. Ed. Engl. 55, 9894-9897. 1,2-ethanedithiol (EDT) was excluded from the cleavage cocktail in this protocol because it reduces the azide of the unnatural amino acid. Briefly, solid-phase peptide synthesis with standard Fmoc conditions was used to synthesize PIP with an unnatural amino acid, 5-azido-I-norvaline, at position 15. In this work, tyrosine was also substituted in place of phenylalanine at position 31 to facilitate peptide detection and concentration measurements by UV absorbance at 280 nm. The method used for HPLC purification was a linear gradient from 10% solvent B to 46% solvent B over 32 min (solvent A, water+0.1% trifluoroacetic acid; solvent B, acetonitrile+0.1% trifluoroacetic acid). A C18 prep column (Microsorb 100-5 C18 Dynamax, 5 μm, 21.4 mm×250 mm; Agilent) was used at a 20 ml min⁻¹ flow rate.

Generation of PIP—GaNAc—PIP—GaINAc Conjugation

Tri-GaINAc-DBCO (5 mg ml⁻¹, 3 equiv.) was added to PIP (1.88 mM in PBS). The reaction was incubated overnight at room temperature and purified by HPLC using an Agilent Infinity Lab Poroshell 120 EC-C19 4.6 nm×50 nm analytical LC column with a linear gradient from 10% solvent B to 46% solvent B over 32 min (solvent A, water+0.1% trifluoroacetic acid; solvent B, acetonitrile+0.1% trifluoroacetic acid) at 0.4 ml min⁻¹.

Antiproliferation Assay

HEPG2 cells (100,000 cells per well in a 24-well plate) were treated with various concentrations of PIP or PIP—GaINAc. After treatments were added, cells were imaged every 4 h for the indicated times in the IncuCyte S3 Live-Cell Analysis system using the phase-imaging channel and a ×10 objective. For co-incubation experiments with ASF, 10 mg ml⁻¹ of ASF (Sigma-Aldrich) was treated with either PIP or PIP—GaINAc. The following analysis parameters were used to quantify phase confluence (%) in the Incucyte software: segmentation adjustment was set to 1, hole fill (μm2) was set to 300 and area minimum (μm²) was set to 150. No other constraints were selected. Percent proliferation was then calculated by normalizing phase confluence values. Specifically, at any given timepoint n in the treatment time course (t=0→t=n), the change in confluence from t=0 to t=n (Δconfluence t=n) was calculated for each well as follows: Δconfluence t=n of well x=(confluence t=n of well x)−(confluence t=0 of well x), where x represents any given well in the experiment. This ensures that the value of each well is set to 0 at t=0. The average Δconfluence at the final time point of the untreated wells was set as the ‘max value’ (equivalent to 100% proliferation). Finally, data were normalized as follows:

% proliferationₜ₌ₙ=n of well x=(Δconfluenceₜ₌ₙ of well x)/(max value),

where it is noted that while the metric is labeled as “% proliferation”, the effects quantitated are the summation of both cell proliferation and cell survival.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1.-64. (canceled)

65. A bifunctional molecule comprising:
a first moiety that specifically binds a cell surface molecule or extracellular molecule, wherein the first moiety is a knottin peptide comprising an engineered loop that binds to the cell surface molecule; and
a second moiety that specifically binds a lysosomal targeting molecule.

66. The bifunctional molecule of claim 65, wherein the knottin peptide is selected from the group consisting of: an EETI-II peptide, an AgRP peptide, a ω-conotoxin peptide, a Kalata B1 peptide, an MCoTI-II peptide, an agatoxin peptide, and a chlorotoxin peptide.

67. The bifunctional molecule of claim 65, wherein the cell surface molecule is a cell surface receptor.

68. The bifunctional molecule of claim 67, wherein the cell adhesion receptor is an integrin.

69. The bifunctional molecule of claim 68, wherein the integrin is selected from the group consisting of: αvβ1 integrin, αvβ3 integrin, αvβ5 integrin, αvβ6 integrin, α5β1 integrin, and any combination thereof.

70. The bifunctional molecule of claim 68, wherein the knottin peptide is an integrin-binding knottin peptide comprising an amino acid sequence having 80% or greater identity to the amino acid sequence set forth in SEQ ID NO:7 or SEQ ID NO:8.

71. The bifunctional molecule of claim 65, wherein the cell surface molecule is present on a cancer cell.

72. The bifunctional molecule of claim 65, wherein the lysosomal targeting molecule is a mannose-6-phosphate receptor (M6PR) or an asialoglycoprotein receptor (ASGPR).

73. A bifunctional molecule comprising:
a first moiety that specifically binds an integrin; and
a second moiety that specifically binds a lysosomal targeting molecule.

74. The bifunctional molecule of claim 73, wherein the integrin is selected from the group consisting of: αvβ1 integrin, αvβ3 integrin, αvβ5 integrin, αvβ6 integrin, α5β1 integrin, and any combination thereof.

75. An in vitro or in vivo method of degrading a cell surface molecule, comprising:
contacting the cell surface molecule with the bifunctional molecule of claim 65 under conditions in which the lysosomal targeting molecule shuttles the cell surface molecule to the lysosome for degradation.

76. An in vitro or in vivo method of degrading an integrin, comprising:
contacting the integrin with the bifunctional molecule of claim 73 under conditions in which the lysosomal targeting molecule shuttles the integrin to the lysosome for degradation.

77. A pharmaceutical composition comprising:
the bifunctional molecule of claim 65; and
a pharmaceutically acceptable carrier.

78. The pharmaceutical composition of claim 77, wherein the pharmaceutical composition is formulated for parenteral administration.

79. A method comprising administering to an individual in need thereof the pharmaceutical composition of claim 77.

80. A method of treating cancer comprising administering to an individual having cancer an effective amount of the pharmaceutical composition of claim 77.

81. A pharmaceutical composition comprising:
the bifunctional molecule of claim 73; and
a pharmaceutically acceptable carrier.

82. The pharmaceutical composition of claim 81, wherein the pharmaceutical composition is formulated for parenteral administration.

83. A method comprising administering to an individual in need thereof the pharmaceutical composition of claim 81.

84. A method of treating cancer comprising administering to an individual having cancer an effective amount of the pharmaceutical composition of claim 81.


SEQUENCE LISTING (42 sequences)

SEQ ID NO: 1 - protein, 28 aa, Ecballium elaterium
GCPRILMRCKQDSDCLAGCVCGPNGFCG

SEQ ID NO: 2 - protein, 34 aa, Homo sapiens
CVRLHESCLGQQVPCCDPAATCYCRFFNAFCYCR

SEQ ID NO: 3 - protein, 29 aa, Unknown
  Kalata B1 peptide
CGETCVGGTCNTPGCTCSWPVCTRNGLPV

SEQ ID NO: 4 - protein, 34 aa, Unknown
  MCoTI-II peptide
SGSDGGVCPKILKKCRRDSDCPGACICRGNGYCG

SEQ ID NO: 5 - protein, 36 aa, Unknown
  chlorotoxin peptide
MCMPCFTTDHQMARKCDDCCGGKGRGKCYGPQCLCR

SEQ ID NO: 6 - protein, 33 aa, Artificial sequence
  synthetic sequence
  SITE (15)..(15): The amino acid at position 15 is an unnatural amino acid (e.g., 5-azido-L-norvaline)
GCPRPRGDNPPLTCXQDSDCLAGCVCGPNGFCG

SEQ ID NO: 7 - protein, 33 aa, Artificial sequence
  synthetic sequence
GCPQGRGDWAPTSCKQDSDCRAGCVCGPNGFCG

SEQ ID NO: 8 - protein, 33 aa, Artificial sequence
  synthetic sequence
  SITE (31)..(31): The amino acid at position 31 is either Phe or Tyr
GCPRPRGDNPPLTCKQDSDCLAGCVCGPNGXCG

SEQ ID NO: 9 - protein, 33 aa, Artificial sequence
  synthetic sequence
GCAEPRGDMPWTWCKQDSDCLAGCVCGPNGFCG

SEQ ID NO: 10 - protein, 33 aa, Artificial sequence
  synthetic sequence
GCVGGRGDWSPKWCKQDSDCPAGCVCGPNGFCG

SEQ ID NO: 11 - protein, 33 aa, Artificial sequence
  synthetic sequence
GCAELRGDRSYPECKQDSDCLAGCVCGPNGFCG

SEQ ID NO: 12 - protein, 33 aa, Artificial sequence
  synthetic sequence
GCRLPRGDVPRPHCKQDSDCQAGCVCGPNGFCG

SEQ ID NO: 13 - protein, 33 aa, Artificial sequence
  synthetic sequence
GCYPLRGDNPYAACKQDSDCRAGCVCGPNGFCG

SEQ ID NO: 14 - protein, 33 aa, Artificial sequence
  synthetic sequence
GCTIGRGDWAPSECKQDSDCLAGCVCGPNGFCG

SEQ ID NO: 15 - protein, 33 aa, Artificial sequence
  synthetic sequence
GCHPPRGDNPPVTCKQDSDCLAGCVCGPNGFCG

SEQ ID NO: 16 - protein, 33 aa, Artificial sequence
  synthetic sequence
GCPEPRGDNPPPSCKQDSDCRAGCVCGPNGFCG

SEQ ID NO: 17 - protein, 33 aa, Artificial sequence
  synthetic sequence
GCLPPRGDNPPPSCKQDSDCQAGCVCGPNGFCG

SEQ ID NO: 18 - protein, 33 aa, Artificial sequence
  synthetic sequence
GCHLGRGDWAPVGCKQDSDCPAGCVCGPNGFCG

SEQ ID NO: 19 - protein, 33 aa, Artificial sequence
  synthetic sequence
GCNVGRGDWAPSECKQDSDCPAGCVCGPNGFCG

SEQ ID NO: 20 - protein, 33 aa, Artificial sequence
  synthetic sequence
GCFPGRGDWAPSSCKQDSDCRAGCVCGPNGFCG

SEQ ID NO: 21 - protein, 33 aa, Artificial sequence
  synthetic sequence
GCPLPRGDNPPTECKQDSDCQAGCVCGPNGFCG

SEQ ID NO: 22 - protein, 33 aa, Artificial sequence
  synthetic sequence
GCSEARGDNPRLSCKQDSDCRAGCVCGPNGFCG

SEQ ID NO: 23 - protein, 33 aa, Artificial sequence
  synthetic sequence
GCLLGRGDWAPEACKQDSDCRAGCVCGPNGFCG

SEQ ID NO: 24 - protein, 33 aa, Artificial sequence
  synthetic sequence
GCHVGRGDWAPLKCKQDSDCQAGCVCGPNGFCG

SEQ ID NO: 25 - protein, 33 aa, Artificial sequence
  synthetic sequence
GCVRGRGDWAPPSCKQDSDCPAGCVCGPNGFCG

SEQ ID NO: 26 - protein, 33 aa, Artificial sequence
  synthetic sequence
GCLGGRGDWAPPACKQDSDCRAGCVCGPNGFCG

SEQ ID NO: 27 - protein, 33 aa, Artificial sequence
  synthetic sequence
GCFVGRGDWAPLTCKQDSDCQAGCVCGPNGFCG

SEQ ID NO: 28 - protein, 33 aa, Artificial sequence
  synthetic sequence
GCPVGRGDWSPASCKQDSDCRAGCVCGPNGFCG

SEQ ID NO: 29 - protein, 33 aa, Artificial sequence
  synthetic sequence
GCPRPRGDNPPLTCKQDSDCLAGCVCGPNGFCG

SEQ ID NO: 30 - protein, 33 aa, Artificial sequence
  synthetic sequence
GCYQGRGDWSPSSCKQDSDCPAGCVCGPNGFCG

SEQ ID NO: 31 - protein, 33 aa, Artificial sequence
  synthetic sequence
GCAPGRGDWAPSECKQDSDCQAGCVCGPNGFCG

SEQ ID NO: 32 - protein, 33 aa, Artificial sequence
  synthetic sequence
GCVQGRGDWSPPSCKQDSDCPAGCVCGPNGFCG

SEQ ID NO: 33 - protein, 33 aa, Artificial sequence
  synthetic sequence
GCHVGRGDWAPEECKQDSDCQAGCVCGPNGFCG

SEQ ID NO: 34 - protein, 33 aa, Artificial sequence
  synthetic sequence
GCDGGRGDWAPPACKQDSDCRAGCVCGPNGFCG

SEQ ID NO: 35 - protein, 33 aa, Artificial sequence
  synthetic sequence
GCPQGRGDWAPEWCKQDSDCPAGCVCGPNGFCG

SEQ ID NO: 36 - protein, 33 aa, Artificial sequence
  synthetic sequence
GCPRGRGDWSPPACKQDSDCQAGCVCGPNGFCG

SEQ ID NO: 37 - protein, 38 aa, Artificial sequence
  synthetic sequence
GCVRLHESCLGQQVPCCDPAATCYCSGRGDNDLVCYCR

SEQ ID NO: 38 - protein, 38 aa, Artificial sequence
  synthetic sequence
GCVRLHESCLGQQVPCCDPAATCYCKGRGDARLQCYCR

SEQ ID NO: 39 - protein, 38 aa, Artificial sequence
  synthetic sequence
GCVRLHESCLGQQVPCCDPAATCYCVGRGDDNLKCYCR

SEQ ID NO: 40 - protein, 38 aa, Artificial sequence
  synthetic sequence
GCVRLHESCLGQQVPCCDPAATCYCEGRGDRDMKCYCR

SEQ ID NO: 41 - protein, 38 aa, Artificial sequence
  synthetic sequence
GCVRLHESCLGQQVPCCDPAATCYCYGRGDNDLRCYCR

SEQ ID NO: 42 - protein, 33 aa, Artificial sequence
  synthetic sequence
  SITE (15)..(15): The amino acid at position 15 is 5-azido-L- norvaline
GCPRPRGDNPPLTCXQDSDCLAGCVCGPNGYCG